SEQUENCE LISTING (3 sequences)

SEQ ID NO: 1 - DNA, 30 bp, Artificial sequence
  Synthetic construct
tatggtaattgtgtgycagmgccgcggtaa

SEQ ID NO: 2 - DNA, 32 bp, Artificial sequence
  Synthetic construct
  misc_feature (20)..(20): n is a, c, g, or t
agtcagccagccggactacnvgggtwtctaat

SEQ ID NO: 3 - DNA, 32 bp, Artificial sequence
  Synthetic construct
aatgatacggcgaccaccgagatctacacgct

(19) United States
(12) Patent Application Publication
(10) Pub. No.: US 2023/0149338 A1
(43) Pub. Date: May 18, 2023

(54) DIAGNOSIS AND TREATMENT OF ADDICTION

(71) Applicant: The Regents of the University of California, Oakland, CA (US)

(72) Inventors: Sierra SIMPSON, San Diego, CA (US); Olivier GEORGE, Lemon Grove, CA (US)

(73) Assignee: The Regents of the University of California, Oakland, CA (US)

(21) Appl. No.: 17/916,610

(22) PCT Filed: Apr. 13, 2021
(86) PCT No.: PCT/US2021/027027

§ 371 Date: Oct. 3, 2022

Related U.S. Application Data
(60) Provisional application No. 63/009,546, filed on Apr. 14, 2020.

(51) Int. Cl. A61K 31/205; A61K 31/19; A61P 25/36

(52) U.S. Cl. CPC A61K 31/205; A61K 31/19; A61P 25/36

(57) ABSTRACT

The present disclosure provides a novel treatment for addiction disorders, particularly opioid addiction. More specifically, the present disclosure provides a treatment for opioid addiction comprising a combination use of any one or more of SCFAs in combination with one or more carnitines, providing effective depletion of microbiome in certain brain regions. A diagnosis method for the progress of the addiction treatment is also provided.

### CROSS-REFERENCE TO RELATED APPLICATIONS

This application claims the benefit of U.S. Provisional Application No. 63/009,546, filed on Apr. 14, 2020, which is incorporated herein by reference in its entirety.

### STATEMENT OF GOVERNMENT INTEREST

This invention was made with government support under grant No. 1U01DA044451 awarded by National Institutes of Health (NIH). The government has certain rights in the invention.

### CROSS REFERENCE TO SEQUENCE LISTING

The genetic components described herein are referred to by sequence identifier numbers (SEQ ID NO). The SEQ ID NOs correspond numerically to the sequence identifiers <400>1, <400>2, etc. The sequence listing in written computer readable format (CRF) submitted Apr. 13, 2021, as a text file named “942103-2030_Sequence_Listing_ST25.txt” created on Mar. 24, 2021, and having a size of 905 bytes, is incorporated by reference in its entirety.

### FIELD OF THE INVENTION

The present disclosure relates generally to diagnosis and treatment of addiction, particularly, opioid addiction.

### BACKGROUND OF THE INVENTION

The United States is currently experiencing a national opioid crisis. More than 2 million Americans currently suffer from substance use disorders that are related to prescription opioid pain relievers, such as oxycodone (National Center for Health Statistics, US Centers for Disease Control and Prevention, CDC/NCHS 2017). Every day, 3,900 people initiate the non-medical use of prescription opioids, and 78 die from overdose (CDC/NCHS 2017). This issue represents a significant health crisis, with high relapse potential and oftentimes a need for life-long opioid replacement therapy. Opioid use disorder is thought to be driven by a cycle of positively reinforcing behaviors and symptoms of negative affect that alter homeostatic mechanisms in the brain, causing tolerance, relapse, and dependence (Koob et al. 1997, Koob 2008, Koob 2009). The prolonged use of opioids is also associated with major side effects, including alterations of microbiome composition by reducing gut motility and inducing constipation, nausea, and vomiting, in addition to several other symptoms of opioid-induced bowel dysfunction (Leppert 2012, Ketwaroo et al. 2013). Chronic opioid use has been shown to alter the gut microbiota in both humans and mice through the expansion of the Gram-positive bacteria Firmicutes and a reduction of Bacteroides (Vinolo et al. 2011, Morrison D 2016).

Opioids alter the microbiome by reducing gut motility. They are often concurrently prescribed with antibiotics, which further reduces the richness and diversity of the resident microbiome. The gut-brain axis has recently emerged as a significant contributor to stress and behavioral alterations, but it has not been explored with regard to opioid use disorder. Concurrent antibiotic and opioid treatment further exacerbate the reduction of alpha diversity (Meng et al. 2015, Le Bastard et al. 2018, Wang et al. 2018). A reduction of these phyla has been shown to reduce microbiome metabolites with important signaling capabilities, such as short-chain fatty acids (den Besten et al. 2013). However, the effects of microbiome depletion on brain regions that are activated by oxycodone intoxication and withdrawal are unknown. This is a critical gap in the literature because changes in brain activation levels that are caused by microbiome depletion may have consequences on neuroadaptations that are produced by chronic oxycodone use, potentially increasing abuse liability.

Addiction is a global health problem. Opioid addiction is particularly problematic. There remains a significant unmet need for a treatment that is non-addictive, is not a replacement therapy, and that can be used when users are still actively using the drug. Any effective intervention is a high priority. The present disclosure addresses these needs.

### SUMMARY OF THE INVENTION

The present disclosure provides that depletion of the microbiome produces widespread region- and state-specific changes in neuronal ensemble activation. Importantly, neuronal ensembles that were altered by antibiotic depletion were in regions of the brain that are involved in opioid use disorder in both intoxication and withdrawal states. The present disclosure represents an important advance in understanding the impact of the gut-brain axis on neuronal recruitment in different drug states and how the microbiome may play a role in opioid use and dependence.

Accordingly, the present disclosure provides a novel treatment of opioid addiction and other diseases and/or addiction disorders. In one aspect, the present disclosure provides a composition having a therapeutically effective amount of a carnitine derivative and a therapeutically effective amount of a short chain fatty acid (SCFA). In certain embodiments, exemplary SCFA compounds are listed as Group A and carnitine compounds are listed as Group B, respectively presented in Tables 1 and 2 below:

TABLE 1
Group A SCFA Compounds

| Lipid | | | | | | | Mass | |
|---|---|---|---|---|---|---|---|---|
| Num- | | | | | | | (g/ | |
| ber | Name | Salt/Ester Name | | | Formula | | mol) | Structure |
| C2:0 | Acetic acid | Ethanoic acid | Acetate | Ethanoate | C₂H₄O₂ | CH₃COOH | 60.05 | 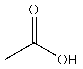 |
| C3:0 | Propionic acid | Propanoic acid | Propionate* | Propanoate | C₃H₆O₂ | CH₃CH₂COOH | 74.08 | 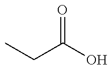 |
| C4:0 | Butyric acid | Butanoic acid | Butyrate* | Butanoate | C₄H₈O₂ | CH₃(CH₂)₂COOH | 88.11 | 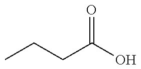 |
| C4:0 | Iboturyic acid | 2- Methylpropanoic acid | Isobutyrate | 2- Methylpropano- ate | C₄H₈O₂ | (CH₃)₂CHCOOH | 88.11 | 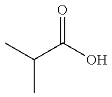 |
| C5:0 | Valeric acid | Pentanoic acid | Valerate | Pentanoate | C₅H₁₀O₂ | CH₃(CH₂)₃COOH | 102.13 | 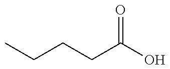 |
| C5:0 | Isovaleric acid | 3- Methylbutanoic acid | Isovalerate | 3- Methylbutanoate | C₅H₁₀O₂ | (CH₃)₂CHCH₂COOH | 102.13 | 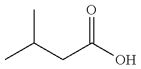 |

*Butyrate, propionate, and combinations of the other SCFAs can potentially be used as treatments.

TABLE 2
Group B Other Molecules from Untargeted Metabolomics

| Compound | | Human Metabolome |
|---|---|---|
| Number | Compound Name | Database Identifier |
| 1 | 2-methylbutyrylcarnitine | HMDB00378 |
| 2 | acetylcarnitine | HMDB00201 |
| 3 | butyrylcarnitine | HMDB02013 |
| 4 | Carnitine (L-carnitine) | HMDB00062 |
| 5 | decanoylcarnitine | HMDB00651 |
| 6 | hexanoylcarnitine | HMDB00705 |
| 7 | isobutyrylcarnitine | HMDB62606 |
| 8 | isovalerylcarnitine | HMDB00688 |
| 9 | lauroylcarnitine | HMDB02250 |
| 10 | myristoylcarnitine | HMDB05066 |
| 11 | octanoylcarnitine | HMDB00791 |
| 12 | palmitoylcarnitine | HMDB00222 |
| 13 | propionylcarnitine | HMDB00824 |
| 14 | stearoylcarnitine | HMDB00848 |
| 15 | valerylcarnitine | HMDB13128 |

A range of potential disorders to be treated is given in Group C presented in Table 3 below.

TABLE 3
Group C Addictions and Disorders to Be Treated

Opioid addiction
Cocaine addiction
Alcohol addiction
Nicotine addiction
Depression
Anxiety
Pain (e.g. chronic pain,
idiopathic pain, neuropathic pain)
Metabolic disorders

Also disclosed are oral dosage forms including the disclosed compositions and at least one pharmaceutically acceptable excipient. In one aspect, the excipient can be selected from a flavoring agent, a coloring agent, a preservative, a disintegrating agent, a coating, a bulking agent, a binder, thickener, a plasticizer, a carrier, or any combination thereof, and the oral dosage form can be a capsule, a tablet, a powder, granules, a liquid, a suspension, an emulsion, a syrup, or any combination thereof.

In one aspect, disclosed herein is a method for treating a disease or a disorder in a subject, the method including administering the disclosed compositions and/or oral dosage forms to the subject. In another aspect, the disease or disorder can be addiction to an opioid, cocaine addiction, alcohol addiction, nicotine addiction, depression, anxiety, pain, a metabolic disorder, or any combination thereof. Exemplary opioids include, but are not limited to, oxycodone, heroin, fentanyl, hydrocodone, morphine, hydromorphone, codeine, methadone, oxymorphone, meperidine, tramadol, carfentanil, buprenorphine, and combinations thereof. Exemplary metabolic disorders include, but are not limited to, type I diabetes, type II diabetes, gestational diabetes, lactose intolerance, fructose malabsorption, galactosemia, glycogen storage disease, or any combination thereof. Exemplary pain types include, but are not limited to, chronic pain, idiopathic pain, neuropathic pain, and combinations thereof. In one aspect, the disease or disorder can be a comorbid psychiatric disorder among subjects addicted to opioids or other substances including, but not limited to, depression, anxiety, pain, or any combination thereof.

In one aspect, when the disease or disorder is addiction, performing the method can prevent or reduce at least one symptom of withdrawal in the subject, and the at least one symptom of withdrawal can be hyperalgesia, changes in appetite, fatigue, irritability, nausea, vomiting, abdominal cramps, diarrhea, tremors, sweating, restlessness, changes in sleeping patterns, headache, nervousness, agitation, depression, aggressive behavior, muscle spasms, seizures, dizziness, confusion, anxiety, or any combination thereof.

The disclosed oral dosage forms can be administered once per day, optionally for a period of at least 6 days up to 12 weeks. In one aspect, the oral dosage forms are administered for 6, 7, 8, 9, or 10 days, or 2, 3, 4, 5, 6, 7, 8, 9, 10, 11, or 12 weeks, or a combination of any of the foregoing values, or a range encompassing any of the foregoing values. The subject can self-administer the oral dosage forms and can be concurrently using one or more opioids, or may not be using one or more opioids. Therapeutically effective amounts of exemplary SCFAs include the following: propionic acid, 200 mg/kg; butyric acid, 350 mg/kg; and acetic acid, 400 mg/kg.

In some aspects, performing the disclosed methods increases a population of at least one microbial species in the subject's gastrointestinal tract such as, for example, a Bacterioides species, a Firmicutes species, a Clostridium species, a Eubacterium species, a Ruminococcus species, a Fusobacterium species, a Burkholderia species, a Butyrivibrio species, a Prevotella species, an Agrobacterium species, a Desulfovibrio species, a Gammaproteobacteria species, an Enterobacteriaceae species, a Eubacteriaceae species, an Anaerofustis species, a Turicibacter species, an Elusimicrobiaceae species, a Bifidobacterium species, a Lactobacillus species, Akkermansia mucinophila, Megasphaera elsdenii, Mitsuokella multiacida, Roseburia intestinalis, Faecalibacterium prausnitzii, Veillonella parvula, Acinetobacter calcoaceticus, Pseudomonas aeruginosa, Biophila wadsworthia, or any combination thereof. In one aspect, the Bacterioides species can be B. efferthii, B. fragilis, or any combination thereof. In another aspect, the Clostridium species can be C. scindens. In still another aspect, the Eubacterium species can be E. hallii, E. dolcichum, or any combination thereof. In one aspect, the Ruminococcus species can be R. bromii, R. obeum, or any combination thereof. In another aspect, the Fusobacterium species can be F. nucleatum. In one aspect, the at least one microbial species can be identified using 16S rRNA sequencing or metagenomics sequencing. In other aspects, performing the method reduces Fos expression in at least one region of the brain such as, for example, the basolateral amygdala, central amygdala, periaqueductal gray matter, locus coeruleus, lateral habenula, paraventricular thalamic nucleus, anterior insula, bed nucleus of the stria terminalus, or any combination thereof. Also disclosed are methods for preventing opioid addiction in subjects using prescribed opioids for pain management and methods for restoring a population of at least one bacterial species in a subject's gastrointestinal tract after the subject has taken antibiotics.

The present disclosure further provides a method of diagnosing addiction in a subject via measurement of taurocholic acid and/or (±)-propionylcarnitine in a biological sample obtained from the subject, wherein the biological sample can be whole blood, plasma, serum, bile, urine, feces, or any combination thereof. In another aspect, the present disclosure provides a method for monitoring the progress of an addiction treatment in a subject, the method comprising comparing the level of taurocholic acid and/or (±)-propionylcarnitine in biological samples from the subject before and during or after initiating the addiction treatment. In any of these aspects, a non-addicted or recovering subject will have higher levels of the metabolites than an addicted subject. In another aspect, the disclosure provides that the metabolite levels can be measured using gas chromatography-mass spectrometry, liquid chromatography-mass spectrometry, nuclear magnetic resonance spectroscopy, enzyme-linked immunosorbent assay (ELISA), or any combination thereof. In a further aspect, the addiction treatment can include administering the disclosed compositions and/or oral dosage forms to the subject.

### BRIEF DESCRIPTION OF THE DRAWINGS

FIGS. 1A-1D. Experimental Design. (FIG. 1A) The rats were made dependent on oxycodone using a passive injection model. The oxycodone treated (OXY) group was not depleted with antibiotics and sacrificed during the intoxication state. The oxycodone and antibiotic treated (OXY+ABX) group was depleted with antibiotics and sacrificed during the intoxication state. The withdrawal (WD) group was not depleted with antibiotics and was sacrificed during naloxone-precipitated withdrawal. The withdrawal and antibiotic treated (WD+ABX) group was sacrificed during the withdrawal state but was depleted with antibiotics. Feces for 16s RNA sequencing were taken at baseline (i.e., before antibiotic or drug exposure). A secondary time point was taken following the experimental paradigm. (FIG. 1B) Timeline for the behavioral tests to confirm the intoxication or withdrawal state (withdrawal scoring and von Frey test). (FIG. 1C) For depletion of the microbiome, the rats were exposed to a cocktail of both Gram-negative and Gram-positive antibiotics in drinking water for 2 weeks before the initiation of dependence. (FIG. 1D) Experimental groups, including both sexes.

FIGS. 2A-2D. Validation of precipitated withdrawal and intoxication in passively injected rats. (FIGS. 2A-2B) The von Frey test was used to confirm that oxycodone withdrawal decreased pain thresholds and oxycodone intoxication increased pain thresholds. No significant difference in pain thresholds was found between the antibiotic-treated and untreated groups. Therefore, each state was pooled. Intoxicated animals exhibited higher pain thresholds compared with the saline and withdrawal groups. The withdrawal group exhibited lower pain thresholds compared with the saline group (p<0.001) and intoxication group (p<0.001). (FIG. 2D) The total withdrawal score was calculated as the sum of all individual withdrawal scores. No difference in withdrawal scores was observed between antibiotic-depleted animals and untreated animals, so each state was pooled (FIG. 2C). The withdrawal animals exhibited higher withdrawal scores compared with intoxicated animals (p<0.001).

FIGS. 3A-3F. Antibiotic depletion reduces alpha diversity. (FIG. 3A) Antibiotic depletion was measured using both the Shannon diversity index and Choa1 index. No difference in either index was found between groups for the initial baseline (BSL) time point. The final time point was taken following both antibiotic depletion and the final oxycodone exposure (post-treatment). In the post-treatment conditions, the OXY+ABX group exhibited a significant decrease in alpha diversity compared with both the saline-treated (SAL) and OXY groups, indicated by the Shannon diversity index (p<0.001) and Chao1 index (p<0.008). (FIG. 3B) A biplot of the principal component analysis by Bray-Curtis similarity clustering indicated that the SAL and OXY groups clustered together, whereas the antibiotic-treated animals clustered separately. (FIG. 3C) Taxonomic plots that show the relative abundance of phyla across different groups at BSL and post-treatment. (FIGS. 3D-3E) The OXY+ABX group exhibited a significant decrease in both Bacteroidetes (p<0.003) and Firmicutes (p<0.001) at the phylum level. Each animal is represented at both time points at the phylum level in the relative abundance plots. (FIG. 3F) Caecal weights were measured at the time of sacrifice. A significant increase in caecal weights was observed in antibiotic-treated rats (p<0.001). *p<0.05, **=p<0.002, ***=p<0.001, significant difference from saline; ^{#}p<0.05, ^{##}=p<0.002, ^{###}=p<0.001 significant difference from OXY group.

FIGS. 4A-4B. Quantification of neuronal ensembles displaying Fos protein expression (Fos+ neurons) during oxycodone intoxication and withdrawal. Ensembles that were recruited during both intoxication and withdrawal were quantified by counting Fos+ neurons that were active 90 min before sacrifice. Saline, saline+naloxone group; OXY, oxycodone+saline; WD, oxycodone+naloxone. Coronal slices (40 μM) were stained for Fos and then visualized using 3,3-diaminobenzidine (DAB) enhanced with nickel to obtain representative images. The representative regions are outlined with dashes. *p<0.05, **=p<0.002, ***=p<0.001, significant difference from saline; ^{#}p<0.05, ^{##}=p<0.002, ^{###}=p<0.001 significant difference from OXY group.

FIGS. 5A-5B. Quantification of Fos+ neuronal ensembles during oxycodone intoxication with antibiotic treatment. (FIG. 5A) Differences in the number of Fos+ neurons were evaluated during oxycodone intoxication between antibiotic-treated and untreated rats. The regions that were altered during intoxication are shown in labeled bars as shown. Coronal slices (40 μM) were stained for Fos and then visualized using DAB enhanced with nickel to obtain representative images. Representative regions are outlined with dashes. (FIG. 5B) Example images of each region that was altered by antibiotic depletion. *p<0.05, **=p<0.002, ***=p<0.001, significant difference from saline; ^{#}p<0.05, ^{##}=p<0.002, ^{###}=p<0.001 significant difference from OXY group.

FIGS. 6A-6B. Quantification of Fos+ neuronal ensembles during oxycodone withdrawal with antibiotic treatment. (FIG. 6A) Differences in the number of Fos+ neurons were evaluated during oxycodone withdrawal between antibiotic-treated and untreated rats. The regions that were altered during withdrawal are represented in by labeled bars as shown. (FIG. 6B) Example images of each region that was altered by antibiotic depletion. *p<0.05, **=p<0.002, ***=p<0.001, significant difference from saline; ^{#}p<0.05, ^{##}=p<0.002, ^{###}=p<0.001 significant difference from non-depleted withdrawal group (WD).

FIG. 7. Correlational analysis of recruited neuronal ensembles. Depletion of the microbiome altered the recruitment of neuronal ensembles during oxycodone intoxication and withdrawal. Correlational analysis of Fos+ cells was performed between anatomically connected structures. Connections with significant correlations (p<0.05) are represented in light gray (positive) and dark gray (negative). Nonsignificant correlations are shown in dark gray with no connecting lines. The regions where recruited neurons significantly increased or decreased upon antibiotic depletion are marked with arrows to indicate the direction of change.

FIG. 8. Co-labeling of G-protein coupled receptors 41 and 43 (GPR41/43) and proto-oncogene cFOS in the basolateral amygdala (BLA), central amygdala (CeA), and periaqueductal gray matter (PAG).

FIG. 9. Antibiotic depletion of the microbiome increases defensive behaviors during withdrawal. SCFA repletion decreases both aggressive and defensive behaviors. *p<0.05, **=p<0.002, ***=p<0.001, significant difference from BSL. ^{#}p<0.05, ^{##}=p<0.002, ^{###}=p<0.001, significant difference from ABX.

FIG. 10. SCFA repletion decreased withdrawal induced hyperalgesia. Following depletion of the microbiome, the ABX time point exhibits decreased pain threshold indicative of withdrawal induced hyperalgesia (p<0.001). Upon SCFA administration, animals exhibit an increase in pain threshold (p<0.001), yet the pain threshold does not return to the level recorded before oxycodone exposure (Naïve) (p=0.044). *p<0.05, ***=p<0.001, significant difference from Naive. ^{###}=p<0.001, significant difference from ABX.

FIG. 11. Heterogenous Stock (HS) rat microbiome depletion experimental design. For this experiment, there are four sampling time points for feces and plasma. Baseline sampling is prior to drug and antibiotic exposure to allow for the innate microbiome to be assessed. The Post-LGA time point is following 21 days of oxycodone self-administration. Following the Post-LGA time point, animals were evenly distributed into three groups based on responding—⅓ of the animals received normal drinking water and ⅔ received the antibiotic cocktail. At the third sampling time point, Post-ABX, animals were again split, ½ of the antibiotic treated animals continued to receive the antibiotic cocktail in the drinking water, and ½ were given the antibiotic cocktail+SCFA. Feces and plasma were again harvested following this time point. This separated the animals into three groups, the OXY group, which received normal drinking water the entire experiment, the OXY+ABX group, which received only the antibiotic cocktail following the Post-LGA time point, and the OXY+ABX+SCFA group, which received all three treatments.

FIG. 12. Antibiotic depletion of the microbiome increases oxycodone self-administration in heterogenous stock rats. Since there is considerable variation in the HS population of rats, responding is plotted as a percent change from an average of the last three days of responding in the long access phase before antibiotics treatment to normalize the magnitude of the observed change for each animal. Animals were considered Responders if there was an increase from the average of the last three days of long access versus the last three days of the antibiotic treatment.

FIGS. 13A-13B. SCFA Responders increase oxycodone self-administration following antibiotic depletion of the microbiome (p<0.003), and reduce intake following SCFA administration compared to the elevated ABX time point (p<0.033). The water group is not significantly different at the matched ABX or SCFA time points (p's>0.05).

FIGS. 14A-14D. Untargeted Metabolomics in Responder and Non-Responder Animals. Responders exhibited reduced levels of (±)-propionylcarnitine in the Responder group (p=0.005) versus the water group. The Non-Responder group exhibited an intermediate phenotype that did not reach significance compared to the water or the Responder group (FIG. 14A). There was a significant difference in the levels of Taurocholic acid (p=0.004). Responders exhibited an increase in Taurocholic acid compared to the water (p=0.009) but not the Non-Responder groups (p=0.12) (FIG. 14B). (FIG. 14C) Example chromatographs with raw traces. A principal component analysis of the untargeted metabolomics results was performed to compare the Responder animals over the time course (FIG. 14D). The PCA demonstrates distinct clusters between each time point. Despite separation of clustering between the baseline and post-SCFA time point, the Responder animals had similar levels of oxycodone intake. While there is some overlay between the Post LGA and Post ABX time points, the antibiotic treatment shifts downward in comparison to the Post-LGA time point.

### DETAILED DESCRIPTION OF THE INVENTION

The present disclosure provides a novel treatment of opioid addiction, and other addiction disorders. In certain embodiments, the present disclosure provides a formulation that include a cocktail of short-chain fatty acids (SCFA) and/or key microbiome resulting metabolites, e.g., carnitine, that can be given p.o. once a day in patients with substance-use-disorders (both abstinent and nonabstinent). The present disclosure further provides a formulation that can be given to patients receiving opioid treatment for pain management in order to limit the adverse effects of opioids on the microbiome. In certain embodiments, the SCFA is listed as Group A, and carnitine is listed as Group B, respectively presented in Tables 1 and 2 above.

Existing treatment options include methadone, buprenorphine, and naltrexone/naloxone. A National Institute on Drug Abuse (NIDA) study found that once treatment is initiated, both a buprenorphine/naloxone combination and an extended release naltrexone formulation are similarly effective in treating opioid use disorder; however, full detoxification is necessary for treatment with naltrexone, which means that initiating treatment among active users can be difficult. Treatment retention can also be difficult as users often relapse. Some of the existing treatments are also habit forming, requiring dosing schedules that are difficult to maintain due to frequent clinic visits. There remains a significant unmet need for a treatment that is non-addictive, not a replacement therapy, and can be used when users are still actively using the drug.

The treatment of the present disclosure fulfills all of those needs. It is non-addictive, can be administered in active users, and does not require regular visits (such as methadone) to administer the treatment. It also reduced withdrawal symptoms such as pain and irritability which are a major impetus for relapse.

A range of disorders to be treated is given in Group C presented above. The present disclosure further provides a method of diagnosing response to addiction treatment via measurement of taurocholic acid.

The present disclosure provides studies identified neuronal ensembles in the BLA, PAG, locus coeruleus (LC), and CeA that were altered by microbiome depletion during intoxication as well as ensembles in the CeA and lateral habenula (LHb) that were altered by microbiome depletion during withdrawal. Additionally, the present disclosure provides evidence that microbiome depletion modulated the recruitment of neuronal ensembles in a state- and region-specific manner. The strongest effects were an increase in Fos activation in the PAG, LC, and CeA during intoxication and a decrease in Fos activation in the CeA during withdrawal. Furthermore, correlational analysis indicated that antibiotic depletion destabilized functional networks that were recruited during intoxication and withdrawal, with a decrease in correlation between brain regions.

The development of oxycodone dependence and state specificity was confirmed by von Frey pain thresholds and withdrawal scoring (Kononoff et al. 2018) after naloxone administration, which demonstrated the development of withdrawal-induced mechanical hyperalgesia and somatic signs of withdrawal. Naloxone-precipitated opioid withdrawal is robust, eliciting vigorous withdrawal symptoms in dependent animals (Gracy 2001). Several brain regions that were recruited during intoxication (BLA) and withdrawal (PAG, CeA, LC, paraventricular thalamic nucleus (PVT), anterior insula (AI), bed nucleus of the stria terminals (BNST), and LHb) were also identified, and oxycodone produced neuroadaptations in key brain regions that are associated with addiction-like behaviors were further confirmed. During oxycodone intoxication, the only brain region that exhibited a significant increase in Fos+ neurons (688%) compared with saline controls was the BLA. The BLA sends projections to the nucleus accumbens (NAc) where it modulates behavioral conditioning and reward valence. This circuit has also been shown to alter operant behaviors through dopaminergic signaling (Wassum et al. 2015). The BLA also has afferent and efferent connections with the CeA, which plays an integral role in anxiety, a distinct player in relapse and the escalation of intake (Tye et al. 2011). Alterations of either of these circuits could result in significant changes in the perception of reward or impact withdrawal symptoms, which could in turn initiate relapse and modify drug-taking behaviors.

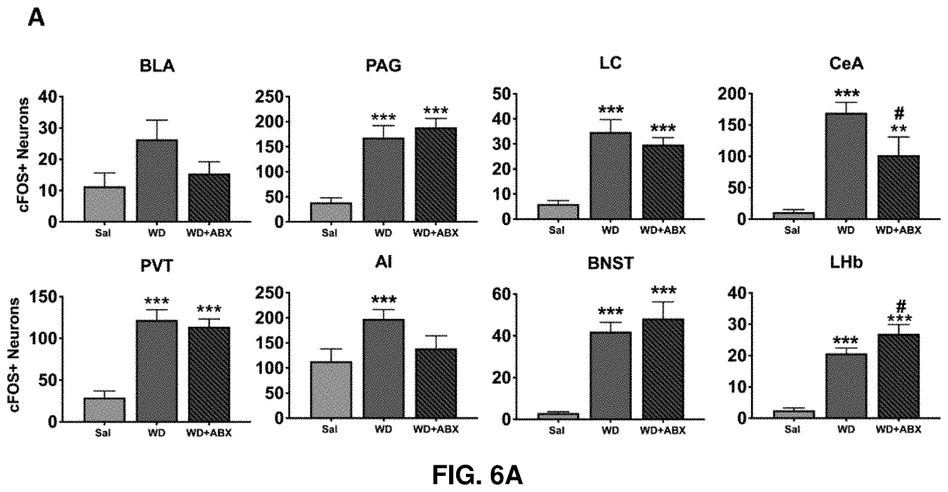

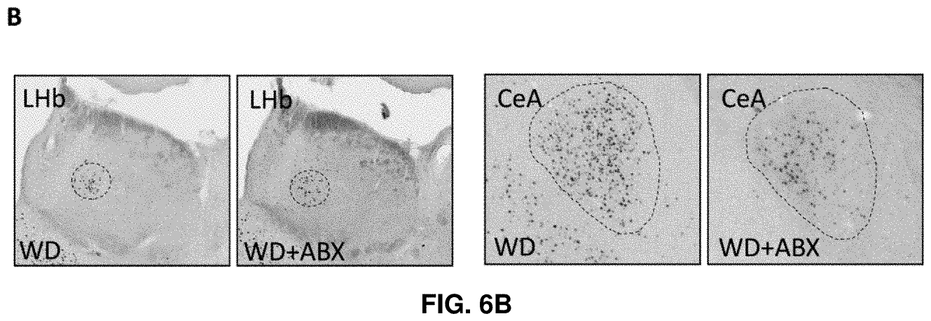

Seven of eight regions of interest in the present disclosure exhibited significantly higher counts of Fos+ neurons during withdrawal compared with controls. These regions (PAG, CeA, LC, PVT, AI, BNST, and LHb) have been shown to be associated with withdrawal-like behaviors, such as anxiety-like behavior, stress-related behaviors, pain, and negative emotional states in general (Koob 2009), all of which are key components in the transition to dependence (Koob et al. 1997, Koob 2008, Koob 2009). For example, one region of the extended amygdala, the lateral BNST (FIGS. 6A-6B), exhibited a 1272% increase in the number of Fos+ neurons in antibiotic-treated animals compared with the control group. This region highly expresses corticotrophin-releasing factor (CRF; (Giardino et al. 2018) and has been implicated in anxiety-like behavior (Avery et al. 2016). Other regions that are involved in withdrawal, such as the PVT, LC, and LHb, exhibited 289%, 475%, and 712% increases, respectively, in the number of Fos+ neurons compared with the control group. These regions have also been shown to be involved in drug withdrawal (Ivanov A et al. 2001, Haight et al. 2014). The LC regulates arousal, responses to stress, and memory. Recruitment of the LC during withdrawal could result in an increase in the salience of each state (Ivanov A et al. 2001, Van Bockstaele et al. 2010). The LHb is important for information processing and valence. This nucleus highly expresses tyrosine hydroxylase, which could result in aberrant dopamine signaling (Hikosaka et al. 2008, Aizawa et al. 2012, Proulx et al. 2014). These results confirmed the validity of the animal model that was used produce opioid intoxication and dependence at both the behavioral and neurobiological levels.

No significant change in behavior was observed between antibiotic-depleted and non-depleted animals, suggesting that these behaviors in this paradigm did not depend on alterations of the microbiome or that no changes was detected because of either ceiling or floor effects during behavioral testing. Indeed, the severity of the somatic signs of withdrawal (1420% increase in withdrawal signs in the WD and WD+ABX groups) and hyperalgesia/allodynia (64% decrease in pain thresholds in the WD and WD+ABX groups) may have masked the contribution of the gut microbiome to these behaviors. Other studies reported that intermittent access to morphine in microbiome-depleted animals was associated with changes in hyperalgesia in the tail withdrawal test (Lee et al. 2018), but this was observed under conditions of 24h spontaneous withdrawal. Moreover, the tail withdrawal test measures sensitivity to a thermal stimulus instead of mechanical allodynia. It may not exclude the possibility that the microbiome may be involved in other behaviors that are related to opioid dependence, including anxiety-like behavior, anhedonia-like behavior, or depression-like behavior. For example, a recent study reported that microbiome depletion exacerbated anxiety-like behavior, which could be rescued by the restoration of a healthy microbiome (Van De Wouw et al. 2018).

Short chain fatty acids function as G-protein coupled receptor ligands for GPR41 and GPR43, as well as act histone deacetylase inhibitors (HDACi). GPR41/GPR43 expression in the basolateral amygdala, periaqueductal gray, medial habenula and the bed nucleus of the stria terminals were characterized. There are also appears to be local circuitry between the basolateral amygdala and the central nucleus of the amygdala.

Regions within the extended amygdala such as the basolateral amygdala, bed nucleus of the stria terminals, and the central amygdala are essential for the negative symptoms of withdrawal. It is believed that SCFA and related metabolites are signaling locally to alter the recruitment of the extended amygdala, as well as regions related to pain (periaqueductal grey). By administering/stabilizing the levels of metabolites, the system from the continuation of negative changes to the homeostatic setpoint of a healthy brain that is often seen in long-term users are protected.

In certain embodiments, the present disclosure provides that depletion of the microbiome alters activation of ensembles recruited during intoxication and withdrawal from opioids. Furthermore, animals supplemented with these metabolites decrease oxycodone self-administration in a clinically relevant translational model of drug use.

Further assessment of the microbiome after antibiotic treatment demonstrated a decrease in both Bacteroidetes and Firmicutes at the phylum level, thus demonstrating the efficacy of antibiotic treatment. The reductions of these phyla have been shown to result in a decrease in circulating short-chain fatty acids (i.e., metabolites that are released by the resident microbiota after the digestion of dietary fiber; (Louis et al. 2017). These metabolites are capable of crossing the blood-brain barrier, signaling through a family of G-protein-coupled receptors (GPR41/GPR43; (Kimura et al. 2011, Inoue et al. 2012, Kimura et al. 2014), and acting as class II histone deacetylate inhibitors (Chen et al. 2003, Waldecker et al. 2008, Fellows et al. 2018), which in turn can alter gene expression profiles. The application of short-chain fatty acids has also been shown to reduce anxiety-like behaviors that are linked to relapse liability in addiction paradigms (van de Wouw et al. 2018, Baxley et al. 2019). Further exploration of the molecular mechanisms by which the microbiome alters the gut-brain axis are necessary to determine the role of microbiome alterations in drug-taking behaviors.

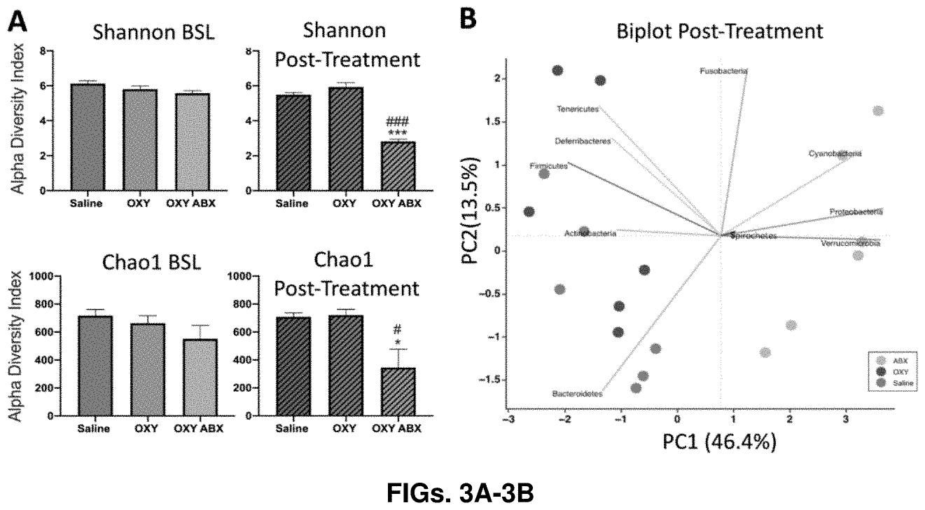

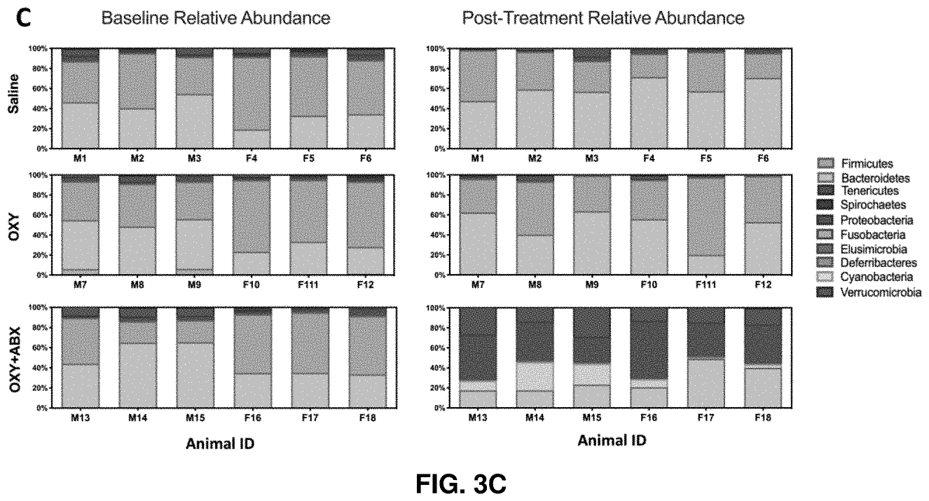

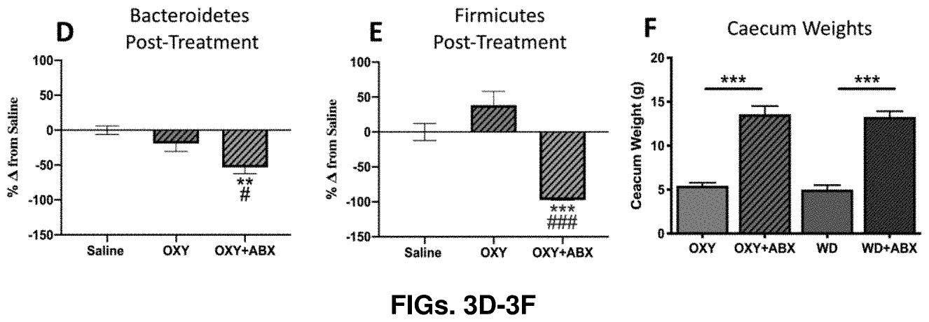

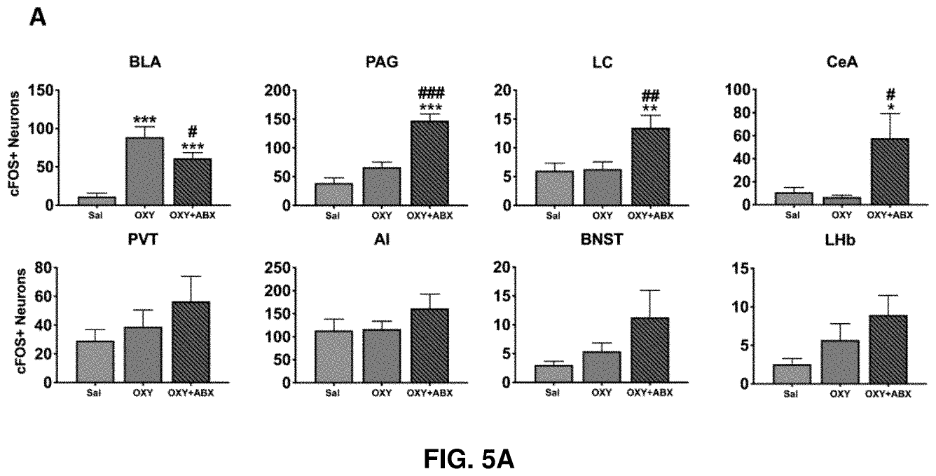

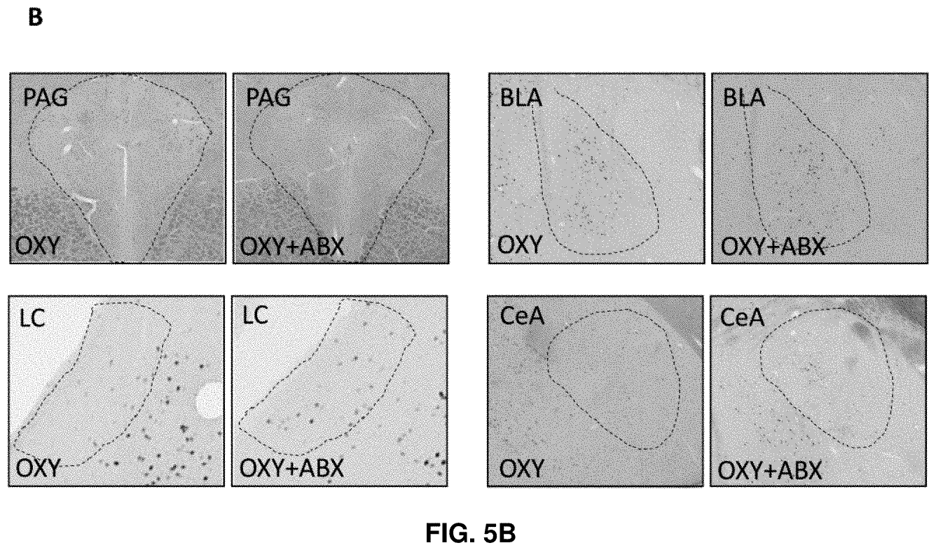

Despite the lack of changes at the behavioral level, the present disclosure provides results showing that antibiotic treatment produced robust changes in how the brain responded to oxycodone intoxication and withdrawal. Compared with the OXY group, the antibiotic-depleted OXY+ABX group exhibited alterations of the recruitment of four regions, including a 122% increase in the PAG, a 119% increase in LC, a 752% increase in the CeA, and a 31% decrease in the BLA. Interestingly, the PAG, LC, and CeA were highly recruited during withdrawal, whereas the BLA was recruited during intoxication (FIGS. 3A-3C). These state- and region-specific changes in Fos+ neuron recruitment represented a major shift in the functional network of these regions, which was confirmed by the correlational analysis (FIGS. 5A-5B). The CeA is associated with negative emotional states and negative reinforcement during withdrawal (George et al. 2007, Koob 2009, de Guglielmo et al. 2016), the activation of which decreased by 40% after antibiotic treatment. The CeA is known to be involved in withdrawal symptoms through the activation of CRF and γ-aminobutyric acid transmission (Funk et al. 2006, de Guglielmo et al. 2019). A significant increase or decrease in neuronal recruitment could result in the desynchronization of ensembles that regulate the perception of negative affective states (Iredale et al. 2001, Contarino et al. 2005, George et al. 2007). Another region that was affected by antibiotic treatment during withdrawal was the LHb, which exhibited a 30% increase in Fos+ neurons. The LHb has been implicated in the processing of emotional valence, depression, and withdrawal from drugs of abuse (Sartorius et al. 2007, Proulx et al. 2014, Shabel et al. 2014). These results indicate that the lack of behavioral changes after antibiotic treatment does not necessarily mean that the brain was not affected by microbiome depletion. Instead, despite the absence of behavioral alterations, the present disclosure provides that antibiotic treatment produced robust alterations of the sensitivity of multiple brain regions that are involved in oxycodone intoxication and withdrawal. This is an important finding that further demonstrates the key role of the microbiome in the modulation of the brain response to drugs. Notably, with a single marker of neuronal reactivity (Fos) that cannot capture the entirety of changes in functional brain networks, additional changes would likely be observed with the use of alternative markers of neuronal reactivity (e.g., Arc and egr1).

To further assess the impact of antibiotic treatment on brain networks, the degree of associations between changes in neuronal activity (i.e., functional connectivity) in each brain region during oxycodone intoxication and withdrawal was evaluated. It was found that antibiotic treatment was associated with dysregulation of the functional network that was produced by decorrelations between several brain regions compared with the control groups. Among the brain regions where functional connections were perturbed, many express opioid peptides and receptors, such as the PAG, LHb, and LC. For example, the OXY group exhibited an overall increase in positive correlations between these brain regions, and these positive correlations were lost after microbiome depletion. A reduction of connectivity was also observed in the WD group, in which the positive correlation between the LHb and BLA was lost. In the PAG, the disruption of connectivity could lead to changes in pain levels, given its importance for pain processing (Chieng et al. 1996) and given that extended opioid use can alter pain thresholds (Chieng et al. 1996). During withdrawal, microbiome depletion also resulted in a shift in functional connectivity of the LC. In the WD group, the LC had a positive correlation with the PVT, whereas the LC had a positive correlation with the PAG in the WD+ABX group.

Activation of the LC is modulated by opioid peptides (Kreibich et al. 2008), CRF, and excitatory amino acids and is well positioned to alter states of intoxication and withdrawal through both connectivity and signaling capability (Van Bockstaele et al. 2006). Overall, these results demonstrate that antibiotic treatment affected the number of Fos+ neurons that were recruited during opioid intoxication and withdrawal and affected the entire functional network of key brain regions that are involved in drug addiction.

Overall, the present disclosure presents the identified brain regions that were activated during oxycodone intoxication and withdrawal and provided evidence that antibiotic-induced depletion of the microbiome modulated the recruitment of neuronal ensembles in a state- and region-specific manner. The results showed that antibiotic treatment produced robust alterations of the sensitivity of multiple brain regions that are involved in oxycodone intoxication and oxycodone withdrawal, despite having no effects on addiction-related behaviors. This is an important finding that further demonstrates the key role of the microbiome in the modulation of the brain response to drugs. Studies that test drug self-administration and behaviorally characterize emotional states could provide alternative readouts of behavioral changes that are associated with microbiome depletion, including drug intake, compulsivity, and anxiety-like behavior. Further cellular and molecular characterizations of specific neuronal populations that are upregulated or downregulated following microbiome depletion and the identification of molecular targets that are modulated by microbiome metabolites are required to better understand the role of the gut-brain axis in addiction and identify possible new treatment targets.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a short chain fatty acid,” “a carnitine derivative,” or “an adjuvant,” includes, but is not limited to, combinations of two or more such short chain fatty acids, carnitine derivatives, or adjuvants, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a short chain fatty acid refers to an amount that is sufficient to achieve the desired improvement in the property modulated by the formulation component, e.g. achieving the desired level of reduction of withdrawal symptoms. The specific level in terms of wt % in a composition required as an effective amount will depend upon a variety of factors including the amount and type of short chain fatty acid, amount and type of carnitine derivative, amount and type of pharmaceutically acceptable excipients, and disorder being treated using the disclosed compositions.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

### Pharmaceutical Compositions

In various aspects, the present disclosure relates to pharmaceutical compositions comprising a therapeutically effective amount of at least one SCFA and a therapeutically effective amount of at least one carnitine derivative. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one SCFA and at least one carnitine derivative as active ingredients, a pharmaceutically acceptable carrier, optionally one or more other therapeutic agents, and optionally one or more adjuvants. The disclosed pharmaceutical compositions include those suitable for oral administration.

In practice, the SCFAs and carnitine derivatives of the present disclosure can be combined as the active ingredient in intimate admixture with a pharmaceutical carrier according to conventional pharmaceutical compounding techniques. Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the SCFAs and carnitine derivatives of the present disclosure can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein and does not have to mix any components together from two or more containers or packages. Typical examples of unit dosage forms are tablets (including scored or coated tablets), capsules or pills for oral administration. This list of unit dosage forms is not intended to be limiting in any way, but merely to represent typical examples of oral unit dosage forms.

The pharmaceutical compositions disclosed herein can comprise an SCFA and a carnitine derivative of the present disclosure as active ingredients, a pharmaceutically acceptable carrier, and optionally one or more additional therapeutic agents. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C12H24O2 to C18H36O2 and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik and angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

### Compositions

In one aspect, disclosed herein are compositions including a therapeutically effective amount of a carnitine derivative and a therapeutically effective amount of a short chain fatty acid.

In another aspect, the carnitine derivative can be L-carnitine, L-carnitine-L-tartrate, 2-methylbutyrylcarnitine, acetylcarnitine, butyrylcarnitine, carnitine, decanoylcarnitine, hexanoylcarnitine, isobutyrylcarnitine, isovalerylcarnitine, lauroylcarnitine, myristoylcarnitine, octanoylcarnitine, palmitoylcarnitine, propionylcarnitine, stearoylcarnitine, valerylcarnitine, or any combination thereof. In one aspect, the therapeutically effective amount of carnitine derivative can be from about 400 mg/day to about 4000 mg/day, or can be about 400, 500, 600, 700, 800, 900, 1000, 1500, 2000, 2500, 3000, 3500, or about 4000 mg/day, or a combination of any of the foregoing values, or a range encompassing any of the foregoing values. In another aspect, the carnitine derivative can be L-carnitine and the therapeutically effective amount is from about 500 mg/day to about 2000 mg/day. In one aspect, the carnitine derivative can be acetylcarnitine and the therapeutically effective amount is from about 600 mg/day to about 3000 mg/day. In still another aspect, the carnitine derivative can be L-carnitine-L-tartrate and the therapeutically effective amount is from about 1000 mg/day to about 4000 mg/day. In an aspect, the carnitine derivative can be propionylcarnitine and the therapeutically effective amount is from about 400 mg/day to about 1000 mg/day.

In still another aspect, the short chain fatty acid can be acetic acid, propionic acid, butyric acid, isobutyric acid, valeric acid, isovaleric acid, or any combination thereof. In one aspect, the therapeutically effective amount of short chain fatty acid can be from about 500 mg/day to about 4000 mg/day, or can be about 500, 600, 700, 800, 900, 1000, 1250, 1500, 1750, 2000, 2250, 2500, 2750, 3000, 3250, 3500, 3750, or about 4000 mg/day, or a combination of any of the foregoing values, or a range encompassing any of the foregoing values.

In one aspect, the short chain fatty acid can be butyric acid or propionic acid and the carnitine derivative can be propionylcarnitine.

In any of these aspects, the composition can be non-addictive.

### Oral Dosage Forms

In one aspect, disclosed herein are oral dosage forms including the disclosed compositions and at least one pharmaceutically acceptable excipient. In another aspect, the pharmaceutically acceptable excipient can be a flavoring agent, a coloring agent, a preservative, a disintegrating agent, a coating, a bulking agent, a binder, thickener, a plasticizer, a carrier, or any combination thereof.

In another aspect, the oral dosage form can be a capsule, a tablet, a powder, granules, a liquid, a suspension, an emulsion, a syrup, or any combination thereof.

### Methods for Treating a Disease or Disorder

In still another aspect, disclosed herein is a method for treating a disease or disorder in a subject, the method including administering the disclosed compositions to the subject. In another aspect, the disease or disorder can be addiction to an opioid, cocaine addiction, alcohol addiction, nicotine addiction, depression, anxiety, a metabolic disorder, or any combination thereof.

In another aspect, the disease or disorder can be addiction to an opioid, cocaine addiction, alcohol addiction, or nicotine addiction, and performing the method prevents or reduces at least one symptom of withdrawal in the subject. In another aspect, the symptom of withdrawal can be hyperalgesia, changes in appetite, fatigue, irritability, nausea, vomiting, abdominal cramps, diarrhea, tremors, sweating, restlessness, changes in sleeping patterns, headache, nervousness, agitation, depression, aggressive behavior, muscle spasms, seizures, dizziness, confusion, anxiety, or any combination thereof.

In any of these aspects, the opioid can be oxycodone, heroin, fentanyl, hydrocodone, morphine, hydromorphone, codeine, methadone, oxymorphone, meperidine, tramadol, carfentanil, buprenorphine, or any combination thereof.

In another aspect, the metabolic disorder can be type I diabetes, type II diabetes, gestational diabetes, lactose intolerance, fructose malabsorption, galactosemia, glycogen storage disease, or any combination thereof.

In one aspect, the composition or oral dosage form can be administered once per day. In another aspect, the composition or oral dosage form can be administered for at least 6 days up to 12 weeks. In one aspect, the oral dosage forms are administered for 6, 7, 8, 9, or 10 days, or 2, 3, 4, 5, 6, 7, 8, 9, 10, 11, or 12 weeks, or a combination of any of the foregoing values, or a range encompassing any of the foregoing values. In another aspect, the composition can be self-administered. In any of these aspects, the subject can be concurrently using one or more opioids, or may not be concurrently using opioids.

In some aspects, the short chain fatty acid can be butyric acid and the therapeutically effective amount can be about 350 mg of butyric acid per kg of body weight of the subject. In another aspect, the short chain fatty acid can be propionic acid and the therapeutically effective amount can be about 200 mg of propionic acid per kg of body weight of the subject. In still another aspect, the short chain fatty acid can be acetic acid and the therapeutically effective amount can be about 400 mg of acetic acid per kg of body weight of the subject.

In one aspect, performing the disclosed method increases a population of at least one microbial species in the subject's gastrointestinal tract. In another aspect, the at least one microbial species can be a Bacterioidetes species, a Firmicutes species, a Clostridium species, a Eubacterium species, a Ruminococcus species, a Fusobacterium species, a Burkholderia species, a Butyrivibrio species, a Prevotella species, an Agrobacterium species, a Desulfovibrio species, a Gammaproteobacteria species, an Enterobacteriaceae species, a Eubacteriaceae species, an Anaerofustis species, a Turicibacter species, an Elusimicrobiaceae species, a Bifidobacterium species, a Lactobacillus species, Akkermansia mucinophila, Megasphaera elsdenii, Mitsuokella multiacida, Roseburia intestinalis, Faecalibacterium prausnitzii, Veillonella parvula, Acinetobacter calcoaceticus, Pseudomonas aeruginosa, Biophila wadsworthia, or any combination thereof. In one aspect, the Bacterioides species can be B. efferthii, B. fragilis, or any combination thereof. In another aspect, the Clostridium species can be C. scindens. In still another aspect, the Eubacterium species can be E. hallii, E. dolcichum, or any combination thereof. In one aspect, the Ruminococcus species can be R. bromii, R. obeum, or any combination thereof. In another aspect, the Fusobacterium species can be F. nucleatum. In one aspect, the at least one microbial species can be identified using 16S rRNA sequencing or metagenomics sequencing.

In one aspect, performing the method reduces Fos expression in at least one region of the brain. In another aspect, the at least one region of the brain can be selected from the basolateral amygdala, central amygdala, periaqueductal gray matter, locus coeruleus, lateral habenula, paraventricular thalamic nucleus, anterior insula, bed nucleus of the stria terminalus, or any combination thereof.

### Methods for Preventing Opioid Addiction

In one aspect, disclosed herein is a method for preventing opioid addiction in a subject, the method including administering the disclosed compositions and/or oral dosage forms to the subject, wherein the subject is currently using one or more prescribed opioids for pain management.

In another aspect, disclosed herein is a method for restoring a population of at least one microbial species in a subject's gastrointestinal tract after the subject has taken antibiotics, the method including administering the disclosed compositions and/or oral dosage forms to the subject. In another aspect, the at least one microbial species can be a Bacterioidetes species, a Firmicutes species, a Clostridium species, a Eubacterium species, a Ruminococcus species, a Fusobacterium species, a Burkholderia species, a Butyrivibrio species, a Prevotella species, an Agrobacterium species, a Desulfovibrio species, a Gammaproteobacteria species, an Enterobacteriaceae species, a Eubacteriaceae species, an Anaerofustis species, a Turicibacter species, an Elusimicrobiaceae species, a Bifidobacterium species, a Lactobacillus species, Akkermansia mucinophila, Megasphaera elsdenii, Mitsuokella multiacida, Roseburia intestinalis, Faecalibacterium prausnitzii, Veillonella parvula, Acinetobacter calcoaceticus, Pseudomonas aeruginosa, Biophila wadsworthia, or any combination thereof. In one aspect, the Bacterioides species can be B. efferthii, B. fragilis, or any combination thereof. In another aspect, the Clostridium species can be C. scindens. In still another aspect, the Eubacterium species can be E. hallii, E. dolcichum, or any combination thereof. In one aspect, the Ruminococcus species can be R. bromii, R. obeum, or any combination thereof. In another aspect, the Fusobacterium species can be F. nucleatum. In one aspect, the at least one microbial species can be identified using 16S rRNA sequencing or metagenomics sequencing.

### Method for Diagnosing Addiction

In one aspect, provided herein is a method for diagnosing addiction to at least one substance in a subject, the method including (a) obtaining a biological sample from the subject; and (b) measuring a level of at least one metabolite in the biological sample. In some aspects, the at least one substance can be an opioid, cocaine, alcohol, nicotine, or any combination thereof. In another aspect, the opioid can be oxycodone, heroin, fentanyl, hydrocodone, morphine, hydromorphone, codeine, methadone, oxymorphone, meperidine, tramadol, carfentanil, buprenorphine, or any combination thereof.

In one aspect, the sample can be whole blood, plasma, serum, bile, urine, feces, or any combination thereof. In another aspect, the at least one metabolite can be (±)-propionylcarnitine, taurocholic acid, or any combination thereof. In still another aspect, a subject having addiction has a lower level of the at least one metabolite compared to a non-addicted subject. In any of these aspects, the level of the at least one metabolite can be measured using gas chromatography-mass spectrometry, liquid chromatography-mass spectrometry, nuclear magnetic resonance spectroscopy, enzyme linked immunosorbent assay (ELISA) or any combination thereof. In one aspect, untargeted metabolomics can be used to compare metabolite levels between a subject with addiction and a non-addicted subject. In another aspect, an unbiased artificial intelligence platform can be used for metabolomic comparisons.

### Method for Monitoring Progress of an Addiction Treatment

In one aspect, disclosed herein is a method for monitoring the progress of an addiction treatment in a subject diagnosed with an addiction, the method including: (a) obtaining a first biological sample from the subject prior to initiating the addiction treatment; (b) measuring a level of at least one metabolite in the first biological sample; (c) initiating the addiction treatment; (d) obtaining a second biological sample from the subject; (e) measuring a level of the at least one metabolite in the second biological sample; and (f) comparing the level of the at least one metabolite in the first biological sample to the level of the at least one metabolite in the second biological sample; wherein an increased level of the at least one metabolite in the second biological sample compared to the level of the at least one metabolite in the first biological sample indicates the addiction treatment is succeeding. In one aspect, untargeted metabolomics can be used to compare metabolite levels between a subject prior to treatment and the subject during or after receiving treatment. In another aspect, an unbiased artificial intelligence platform can be used for metabolomic comparisons.

In any of these aspects, the first biological sample and the second biological sample can be whole blood, plasma, serum, bile, urine, feces, or any combination thereof. In another aspect, the at least one metabolite can be (±)-propionylcarnitine, taurocholic acid, or any combination thereof. In any of these aspects, the level of the at least one metabolite can be measured using gas chromatography-mass spectrometry, liquid chromatography-mass spectrometry, nuclear magnetic resonance spectroscopy, enzyme linked immunosorbent assay (ELISA), or any combination thereof.

In another aspect, the addiction treatment can include administering the disclosed compositions and/or oral dosage forms to the subject.

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### EXAMPLES

To test that changes in the microbiome affect the brain's response to oxycodone intoxication and withdrawal, the effect of depletion of the microbiome was investigated using a non-absorbable antibiotic cocktail for 2 weeks (Kiraly et al. 2016) on the level of activation of brain regions that are recruited during intoxication and withdrawal using the immediate early gene c-fos as a measure of neuronal activity (Bullitt 1990, Koya et al. 2012, Chung 2015). A translationally relevant animal model of oxycodone dependence that is characterized by chronic daily injections of oxycodone was used (Wiebelhaus et al. 2015). Successful depletion of the microbiome was assessed by 16S RNA sequencing and downstream alpha diversity analysis using the Shannon diversity index and Choa1 index with caecal size as a secondary measure (Kiraly et al. 2016, Ge et al. 2017, Kennedy et al. 2018). Microbiome depletion was characterized by a significant decrease in both Bacteroidetes and Firmicutes.

These phyla are estimated to constitute 80-90% of the resident microbiota (Brooks et al. 2003, Belheouane et al. 2017). Neuronal ensemble analysis was focused on brain regions that are known to be involved in oxycodone intoxication and withdrawal, including regions in the extended amygdala (e.g., basolateral amygdala [BLA], central nucleus of the amygdala [CeA], and bed nucleus of the stria terminals [BNST]), brain regions that are involved in the regulation of stress and pain processing (e.g., locus coeruleus [LC], paraventricular nucleus of the thalamus [PVT], and periaqueductal gray [PAG]), and brain regions that are involved in craving/reward processing (e.g., agranular insular cortex [AI] and lateral habenula medial parvocellular part [LHb]).

### Example 1

### Materials and Methods

### Experimental Design

Oxycodone dependence was initiated using a passive injection model, in which rats were injected subcutaneously with oxycodone (2 mg/kg) every 12 h for 5 days, 2 weeks after microbiome depletion or water treatment. The rats were subjected to microbiome depletion using a cocktail of non-absorbable antibiotics in their drinking water. Control rats were given regular drinking water. Prolonged oxycodone administration leads to tolerance and physical dependence, demonstrated by withdrawal symptoms upon the cessation of drug administration. Withdrawal was precipitated at the end of the injection paradigm with a subcutaneous injection of naloxone (1 mg/kg) in the saline (SAL) group, withdrawal (WD) group, and antibiotic-treated withdrawal (WD+ABX) group. For the intoxication state, the rats were given a saline injection at the same time as the naloxone group. The OXY group included animals that were not treated with antibiotics in the intoxicated state, and the OXY+ABX group included animals that were treated with antibiotics in the intoxicated state. Withdrawal scores were taken following the naloxone injection to confirm the withdrawal state. von Frey pain threshold tests were performed 2 h following the oxycodone injection to confirm the intoxication state and oxycodone-induced analgesia. von Frey pain threshold tests were also performed following naloxone injections in the SAL, WD, and WD+ABX groups to confirm withdrawal-induced hyperalgesia. The rats were sacrificed and perfused 90 min after the naloxone injection in the WD, WD+ABX, and SAL groups and after 90 min following the saline injection (matched with the naloxone injection time point) in the OXY and OXY+ABX groups. Brains were then cryopreserved and stained for Fos expression.

### Subjects

Adult male (n=22) and female (n=25) Sprague-Dawley rats were housed in groups of three under a 12 h/12 h light/dark cycle (light on at 10:00 PM) in a humidity-controlled vivarium with ad libitum access to tap water and food pellets (P J Noyes, Lancaster, N.H., USA). All of the procedures were conducted in strict adherence to the National Institutes of Health Guide for the Care and Use of Laboratory Animals and were approved by the Institutional Animal Care and Use Committee of the [author institution]. At the time of testing, the subjects' body weight ranged between 280 and 350 for females and 350 to 400 g for males.

### Drugs

Oxycodone (Sigma Aldrich, St. Louis, Mo., USA) was dissolved in 0.9% sodium chloride (Hospira, Lake Forest, Ill., USA) and administered subcutaneously (2 mg/kg) every 12 h. Naloxone (Sigma Aldrich, St. Louis, Mo., USA) was dissolved in 0.9% sodium chloride (Hospira, Lake Forest, Ill., USA) and administered subcutaneously (1 mg/kg). Antibiotic doses were given at the following concentrations in drinking water according to a previous study (Kiraly et al. 2016): bacitracin (0.5 mg/mL), neomycin (2 mg/mL), vancomycin (0.2 mg/mL), and pimaricin (1.2 μg/mL). These antibiotics were selected because they are absorbed in the intestine and have been previously reported to only have efficacy in depleting the microbiome when administered orally. The antibiotic mixture was changed every 2 days. The rats were weighed weekly to ensure the maintenance of body weight.

### Mechanical Nociceptive Von Frey Test

Mechanical nociception, reflected by hindpaw withdrawal thresholds, was determined by an observer who was blind to the experimental groups using von Frey filaments that ranged from 3.63 to 281.838 g as previously reported (Dixon 1965, Kononoff et al. 2018). A test session began after 10 min of habituation to the testing environment immediately following the subcutaneous injection of naloxone (WD, WD+ABX, and SAL groups) or saline (OXY and OXY+ABX groups). A series of von Frey filaments were applied from below the wire mesh to the central region of the plantar surface of the left and right hindpaws in ascending order, beginning with the smallest filament (3.63 g). The filament was applied until buckling occurred, and it remained in place for approximately 2 s. A sharp withdrawal of the hindpaw indicated a positive response. The stimulus was incrementally increased until a positive response was observed and then decreased until a negative response was observed according to the statistical up-down method of (Dixon 1965). The 50% paw withdrawal threshold was determined by the formula Xf+kδ, where Xf is the last von Frey filament applied, k is the Dixon value that corresponded to the response pattern, and 5 is the mean difference between stimuli. Once the threshold was determined for the left hindpaw, the same testing procedure was repeated for the right hindpaw after 5 min. Paw withdrawal thresholds were recorded at baseline, during intoxication, and during precipitated withdrawal.

### Withdrawal Score

To precipitate withdrawal, the rats were subcutaneously injected with naloxone and then placed in a Plexiglas observation chamber. The rats were observed for 30 min for signs of withdrawal by three independent observers who were blind to the experimental conditions.

Withdrawal symptoms were categorized using a scale that was modified from (Gellert et al. 1978) and (Cicero et al. 2002). Graded signs of withdrawal were scored as the following: wet dog shakes (1 or 2 shakes=2; 3 or 4 shakes=3; ≥4 shakes=4) and escape attempts (2-4 attempts=1; 5-9 attempts=2; ≥10 attempts=3). The following behaviors were measured per instance: abnormal posture/writhing, teeth chattering, ptosis (drooping eyelids), diarrhea, profuse salivation, urination, swallowing movements, and chromodacyorrhea (red tears). The total withdrawal score was calculated as the sum of all of the individual withdrawal scores. Immediately after withdrawal testing, all of the rats were returned to standard cages and given unrestricted access to food and water.

### Immunohistochemistry

The rats were injected with oxycodone (2 mg/kg) on the sixth day of the dosing schedule. Two hours later, the rats were injected with either saline (OXY and OXY+ABX groups) or 1 mg/kg naloxone (WD, WD+ABX, and SAL groups) to maintain intoxication or precipitate withdrawal, respectively. Brains were collected 90 min after the injection of naloxone or saline to produce both intoxicated and withdrawal groups. The rats were deeply anesthetized and perfused with 100 mL of phosphate-buffered saline (PBS) followed by 100 mL of 4% paraformaldehyde (PFA). Brains were postfixed in 4% PFA overnight and transferred to 30% sucrose in a PBS/0.1% azide solution at 4° C. for 2 days. Brains were frozen in powdered dry ice for 15-30 s and then sectioned coronally at 40 μm thickness throughout the brain using a cryostat. Representative sections were quantified from the following regions at the following coordinates according to the (Paxinos 2007) rat brain atlas: PVT (bregma −3.36), LHb (bregma −3.36), BLA (bregma −3.12), CeA (bregma −3.12), PAG (bregma −5.76), anterior agranular insula bregma (+3.00), BNST (bregma 0.00), and LC (bregma −9.16).

### 3,3′-Diaminobenzidine Staining

The sections were washed in PBS for 10 min three times and then incubated in 1% H2O2/PBS for 20 min to quench endogenous peroxidase activity. The sections were then rinsed for 10 min three times in PBS and incubated in blocking solution (PBS+0.3% TritonX-100, 1 mg/mL bovine serum albumin, and 5% normal donkey serum) for 1 h. The sections were then incubated for 24 h at room temperature with rabbit monoclonal anti-Fos antibody (Cell Signaling Technologies, catalog no. 2250, RRID: AB_2247211) diluted 1:1000 in PBS/0.5% Tween-20 and 5% normal donkey serum. Following incubation with the primary antibody, the sections were washed for 10 min three times in PBS and incubated for 2 h in undiluted rabbit ImmPress horseradish peroxidase reagent (Vector Laboratories, catalog no. MP-7451, RRID: 2631198).

The sections were then washed in PBS for 10 min three times and then developed for 6 min in Vector peroxidase 3,3′-diaminobenzidine (DAB) substrate (Vector Laboratories, catalog no. SK-4100, RRID: 2336382) enhanced with nickel chloride. Following rinses in PBS for 10 min three times, the sections were mounted on Fisher Super Frost Plus slides, air dried, and coverslipped with PVA-DABCO (Sigma Aldrich, catalog no. 10981). Following coverslipping, images were acquired using a Keyence BZX700 fluorescent microscope and analyzed using Fiji software.

### Imaging and Quantification

Quantitative analysis to obtain unbiased estimates of the total number of Fos+positive cell bodies was performed using Fiji software with the Analyze Particles module (Schindelin et al. 2012). Slides were imaged on a Keyence BZX700 slide scanner and stitched at 2□ magnification. Three sections were bilaterally analyzed for each rat and then averaged per animal (counted as n=1). A total of 4-6 animals of each sex per group were analyzed. Cell numbers were normalized to the area of the region of interest.

### Connectivity Analysis

Functional connections between brain regions were determined by calculating interregional Pearson correlations for each treatment (n=8-10/group, two-tailed p<0.05 for positive and negative connectivity). The statistical analyses were performed using Statistica and GraphPad Prism 7 software.

### 16S RNA Sequencing

At each time point, approximately two fecal pellets were harvested and put directly into a cryovial, which was placed on powered dry ice. The pellets were then stored at −80° C. until processing for sequencing. At the time of DNA extraction, feces were thawed and extracted with the Qiagen DNeasy powersoil kit. The samples are then amplified by PCR in triplicate and then pooled. The 16S V4 gene was amplified using universal primers (525F-806R). Each sample was normalized to 240 ng per sample and purified. After purification, the A260/A280 ratio of the final pool was recorded to ensure purity, with a tolerance range of 1.8-2.0. The barcoded amplicons from all of the samples were normalized, pooled to construct the sequencing library, and sequenced using an Illumina MiSeq sequencer. The sequencing primers were the following:

forward
(SEQ ID NO: 1)
(TATGGTAATTGTGTGYCAGMGCCGCGGTAA),
reverse
(SEQ ID NO: 2)
(AGTCAGCCAGCCGGACTACNVGGGTWTCTAAT),
and
index sequence
(SEQ ID NO: 3)
(AATGATACGGCGACCACCGAGATCTACACGCT).

After sequencing, the raw files were prepared, filtered for quality, and demultiplexed. Operational taxonomic units (OTUs) were selected using open-reference OUT picking based on 97% sequence similarity to the Greengenes database. Taxonomy assignment and rarefaction were performed using Qiita with 15,000 reads per sample (Gonzalez et al. 2018). The alpha diversity was measured using both the Shannon diversity index and Chao1 index and compared between baseline and treatment conditions. Bray-Curtis dissimilarity clustering analysis was performed for principal component analysis (PCoA) to generate the biplot.

### Statistical Analysis

The data are expressed as mean±SEM. The von Frey test data were first analyzed using a mixed-model analysis of variance (ANOVA), with group (OXY or WD) and treatment (water or ABX) as the between-subjects factor and time point (baseline or withdrawal/intoxication) as the within-subjects factor. For these measures, no difference was found between antibiotic-treated and water-treated animals. Therefore, the groups were pooled for the state (intoxication or withdrawal) factor. Follow-up Student-Newman-Keuls post hoc tests were performed to assess effects of group at each time point when a significant interaction was revealed. For withdrawal scores, microbiome assessments (Shannon diversity index, Chao1 index, Bacteroidetes Δ, Firmicutes Δ), caecum weights, and the recruitment of Fos+ neuronal ensembles, one-way ANOVA was performed, followed by the Student-Newman-Keuls post hoc test. No differences in withdrawal scores were found between antibiotic-treated animals and untreated animals. Therefore, the groups were pooled for the state (intoxication or withdrawal) factor and analyzed using one-way ANOVA, followed by the Student-Newman-Keuls post hoc test. The data were analyzed using either Statistica 7 software (correlational analysis) or GraphPad Prism 7 software (ANOVA). Values of p<0.05 were considered statistically significant. All of the statistical results are detailed in the statistical table.

### Example 2

### Repeated Passive Oxycodone Injections Induced Intoxication, Dependence, and Withdrawal Behaviors

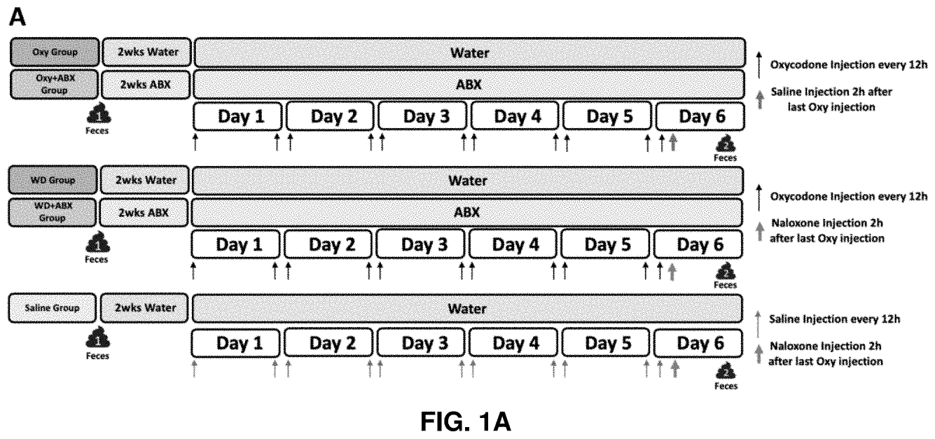

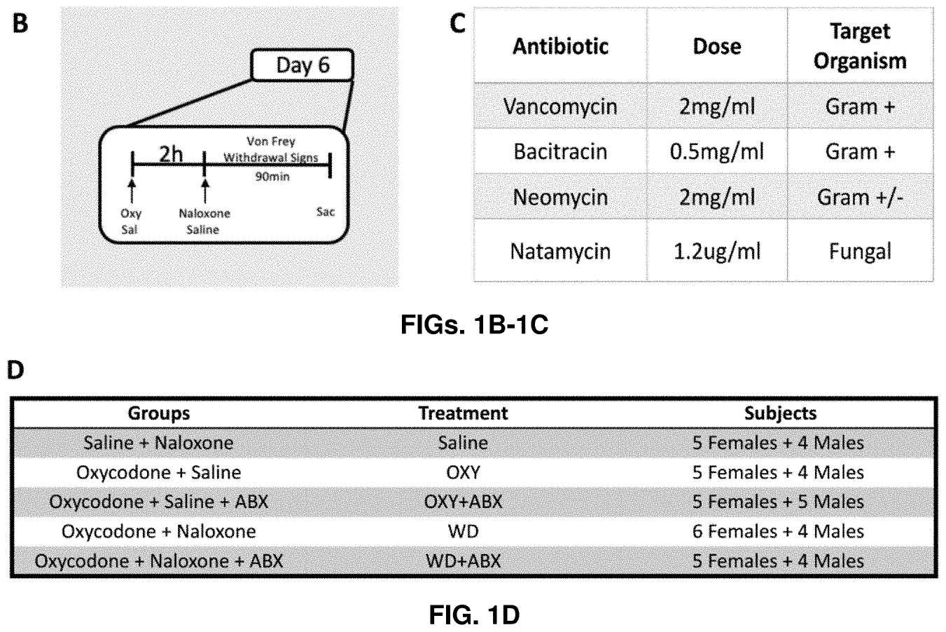

Prior to initiating dependence, the rats (n=47 [22 males, 25 females]) were given either water only or water that was mixed with a non-absorbable antibiotic cocktail for 2 weeks to deplete a significant portion of the microbiome as previously reported (Kiraly et al. 2016). Significant perturbation of the innate microbiome is necessary to reduce the resident richness and diversity of the microbial milieu (Reikvam et al. 2011). The rats were then given twice-daily injections of either oxycodone (2 mg/kg for 5 days) to induce dependence or saline as a control. On the sixth day, the rats received a naloxone injection (1 mg/kg) to precipitate withdrawal or a saline injection to maintain the state of intoxication. All of the antibiotic-treated groups were maintained on the treatment throughout the entire testing period. For a diagram of the experimental design, see FIG. 1A. A behavioral testing timeline for the final day is presented in FIG. 1B.

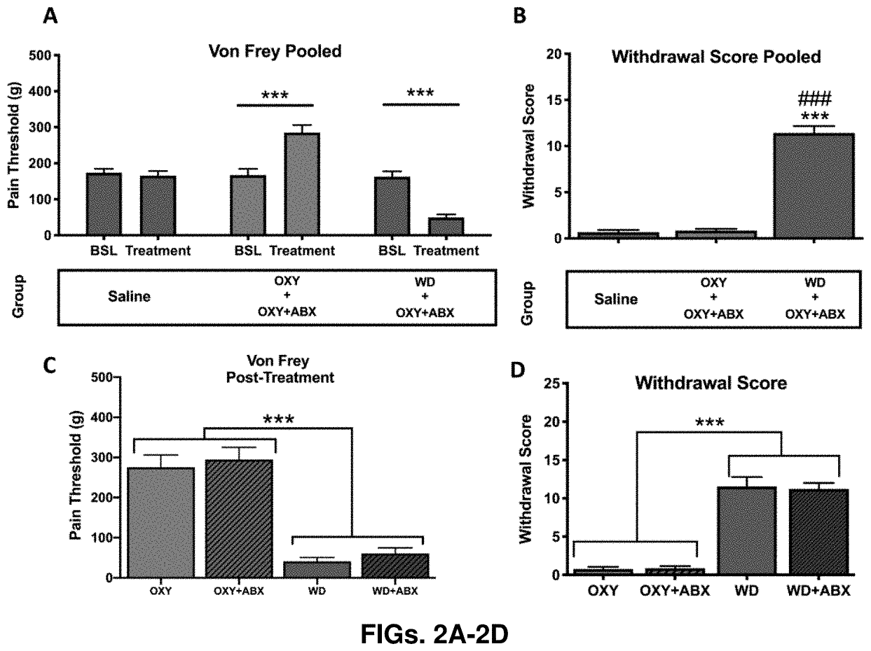

Paw withdrawal thresholds were examined using the von Frey test to evaluate mechanical nociception (OXY and OXY+ABX groups) and withdrawal-induced hyperalgesia/allodynia (WD, WD+ABX, and SAL groups; (Dixon 1965, Kononoff et al. 2018). Intoxicated animals had significantly higher von Frey scores compared with baseline scores, and animals in withdrawal had significantly lower von Frey scores compared with baseline scores and intoxicated animals (F2,44=41.0, p<0.001; FIG. 2A). The one-way ANOVA revealed that at the intoxication/withdrawal time point, all of the groups significantly differed from one another, such that intoxicated animals had significantly higher von Frey scores and withdrawal animals had significantly lower von Frey scores compared with all of the other groups (all p<0.001; FIGS. 2A-2B).

Animals that underwent withdrawal had higher withdrawal scores compared with intoxicated animals, regardless of treatment (F2,44=123.3, p<0.001). A significant main effect of group was observed, in which animals in the withdrawal condition had significantly higher withdrawal scores compared with both saline and intoxicated animals (both p<0.001). No significant difference in withdrawal scores was found between saline and intoxicated animals (p >0.99).

### Example 3

### Antibiotic Depletion of the Microbiome Decreased Alpha Diversity and Increased Caecal Size

To confirm that the microbiome was depleted by antibiotics, alpha diversity of the samples was assessed based on the Shannon diversity index and Chao1 index. The Chao1 index qualitatively measures alpha diversity but gives more weight to rare species. The Shannon diversity index accounts for both richness and evenness to determine alpha diversity. The one-way ANOVA revealed that antibiotic treatment significant decreased alpha diversity (F2,15=87.15, p<0.001; FIG. 3A). The Student-Newman-Keuls post hoc test revealed no difference in the Shannon diversity index between the SAL and OXY groups (p<0.220), whereas the Shannon diversity index significantly decreased in the OXY+ABX compared with both the water group (p<0.001) and OXY group (p<0.001). Similarly, the Chao1 test revealed a decrease in alpha diversity in antibiotic-treated animals (F2,15=6.873, p<0.008). The Student-Newman-Keuls post hoc test showed no difference in alpha diversity between SAL and OXY groups (p<0.993), whereas alpha diversity decreased in the OXY+ABX group compared with both the SAL group (p<0.017) and OXY group (p<0.014). To ensure the absence of initial differences between groups, the groups were compared at baseline (i.e., before antibiotic or drug exposure). No difference in either Shannon diversity index (F2,15=2.745, p<0.096) or Choa1 index (F2,15=1.52, p<0.251) was observed (FIG. 3A).

A principal component analysis of fecal samples was performed to compare the SAL, OXY, and OXY+ABX groups. The SAL and OXY groups clustered together, whereas the OXY+ABX group clustered separately (FIG. 3B). At the phylum level, Bacteroidetes and Firmicutes were the most abundant under baseline conditions, accounting for 40.33% and 50.30% of the resident microbiota, respectively (FIG. 3C). After treatment, a decrease in Bacteroidetes was observed in the OXY group (−19.01%) and OXY+ABX group (−53.44%) compared with the matched SAL group. A decrease in Firmicutes was observed after treatment in the OXY+ABX group (−97.3%), whereas the OXY group exhibited an increase in Firmicutes following oxycodone exposure (38.5%) compared with the matched saline group. The OXY+ABX group exhibited increases in Cyanobacteria, Proteobacteria, and Verrucomicrobia compared with the SAL and OXY groups (FIG. 3C).

The shift in microbial communities was further analyzed by measuring changes in Bacteroidetes (FIG. 3D) and Firmicutes (FIG. 3E) relative to the matched saline group after treatment. Bacteroidetes significantly decreased following antibiotic treatment (F2,15=8.844, p<0.003). The Student-Newman-Keuls post hoc test revealed a significant decrease in Bacteroidetes in the OXY+ABX group compared with the water group (p<0.002) and the OXY group (p<0.03). No difference in Bacteroidetes was found between the OXY and SAL groups (p >0.05). A similar decrease in Firmicutes was observed after antibiotic treatment (F2,15=27.43, p<0.003). The Student-Newman-Keuls post hoc test revealed a significant decrease in Firmicutes in the OXY+ABX group compared with the SAL group (p<0.001) and the OXY group (p<0.001). No difference in Firmicutes was found between the OXY and SAL groups (p>0.05).

As previously reported, gnotobiotic mice and mice with antibiotic depletion exhibited an increase in caecal size. In the present example, animals that received antibiotic treatment had significantly heavier caeca weights compared with animals that did not receive antibiotic treatment, regardless of whether they were in intoxicated or withdrawal conditions (F3,34=55.34, p<0.001; FIG. 3F). No difference in caecal weight was observed among OXY or WD group compared with saline animals under water treatment conditions (p>0.73).

### Example 4

### Characterization of the Recruitment of Fos+ Neuronal Ensembles of Oxycodone Intoxication and Withdrawal

Basolateral amygdala. The ANOVA revealed a significant effect of group on the number of recruited Fos+ neurons (F2,26=22.3, p<0.001). The Student-Newman-Keuls post hoc test revealed significantly fewer Fos+ neurons in the SAL group compared with the OXY group. The WD group exhibited a significant decrease in Fos+ neurons compared with the OXY group but no difference from the SAL group.

Central nucleus of the amygdala. The ANOVA revealed a significant effect of group on the number of recruited Fos+ neurons (F2,26=73.97, p<0.001). The Student-Newman-Keuls post hoc test revealed no difference between the SAL and OXY groups but a significant difference between the SAL and WD groups and between the OXY and WD groups.

Locus coeruleus. The ANOVA revealed a significant effect of group on the number of recruited Fos+ neurons (F2,26=25.2, p<0.001). The Student-Newman-Keuls post hoc test revealed a significant difference between the SAL group and the WD and WD+ABX groups and between the OXY group and WD and WD+ABX groups.

Periaqueductal gray. The ANOVA revealed a significant effect of group on the number of recruited Fos+ neurons (F2,26=16.9, p<0.001). The Student-Newman-Keuls post hoc test revealed a significant increase in Fos+ neurons in the WD group compared with the SAL and OXY groups.

Lateral habenula medial parvocellular part. The ANOVA revealed a significant effect of group on the number of recruited Fos+ neurons (F2,26=37.3, p<0.001). The Student-Newman-Keuls post hoc test revealed a significant increase in Fos+ neurons in the WD group compared with the SAL and OXY groups. No difference in the number of Fos+ neurons was found between the SAL and OXY groups.

Periventricular thalamic nucleus. The ANOVA revealed a significant effect of group on the number of recruited Fos+ neurons (F2,26=23, p<0.001). The Student-Newman-Keuls post hoc test revealed a significant increase in Fos+ positive neurons in the WD group compared with the SAL and OXY groups. No difference in the number of Fos+ neurons was found between the SAL and OXY groups.

Bed nucleus of the stria terminals. The ANOVA revealed a significant effect of group on the number of recruited Fos+ neurons (F2,26=55.7, p<0.001). The Student-Newman-Keuls post hoc test revealed a significant increase in the number of Fos+ neurons in the WD group compared with the SAL and OXY groups. No significant difference in the number of Fos+ neurons was found between the SAL and OXY groups.

Anterior insula. The ANOVA revealed a significant effect of group on the number of recruited Fos+ neurons (F2,26=5.92, p=0.008). The Student-Newman-Keuls post hoc test revealed a significant increase in the number of Fos+ neurons in the WD group compared with the SAL and OXY groups.

### Example 5

### Characterization of the Effect of Antibiotic Depletion on the Recruitment of Fos+ Neuronal Ensembles of Oxycodone Intoxication and Withdrawal

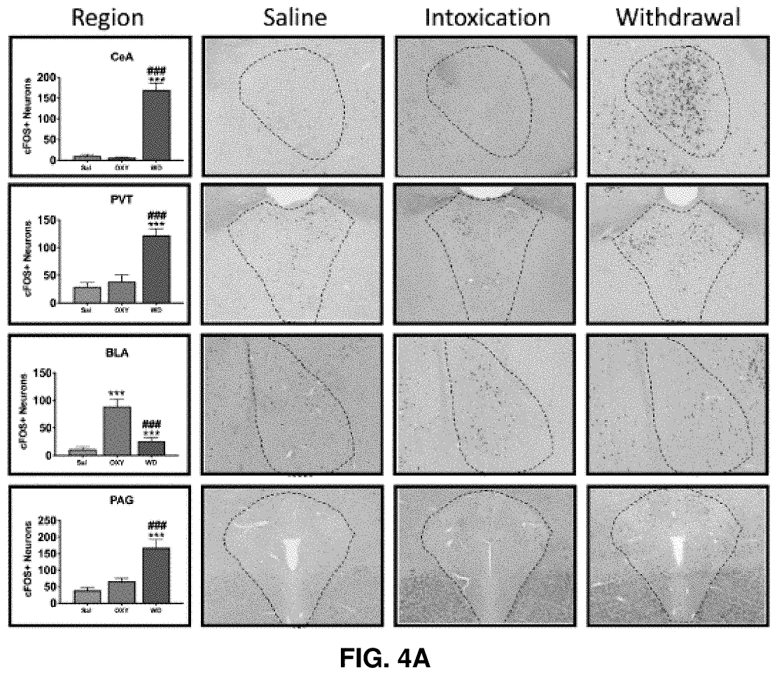

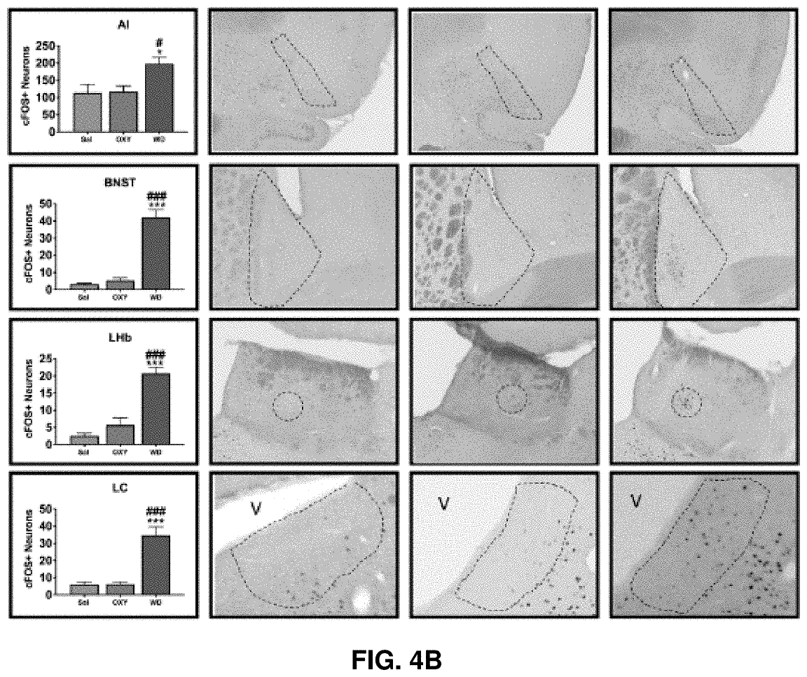

Basolateral amygdala. The ANOVA revealed a significant effect of treatment on the number of recruited Fos+ neurons in the OXY group (F2,24=18.8, p<0.001; FIG. 5A). The Student-Newman-Keuls post hoc test revealed a significant decrease in the number of Fos+ neurons in the OXY group compared with the OXY+ABX group. Example DAB images are shown in FIG. 5B, example images of the untreated, matched OXY group are repeated from FIGS. 4A-4B for comparisons to the antibiotic-treated groups. There was no significant difference in the number of Fos+ neurons in the withdrawal group (WD and WD+ABX) (F2,26=2.45, p<0.106; FIG. 6A).

Central nucleus of the amygdala. The ANOVA revealed a significant effect of treatment on the number of recruited Fos+ neurons in the OXY group (F2,24=5.13, p<0.014) and WD group (F2,26=17.9, p<0.001). The Student-Newman-Keuls post hoc test revealed a significant decrease in the number of Fos+ neurons in the WD group compared with the WD+ABX group and a significant increase in the number of Fos+ neurons in the OXY group compared with the OXY+ABX group. Example DAB images are shown in FIGS. 5B and 6B. Example images of untreated, matched groups are repeated from FIGS. 4A-4B for comparisons to the antibiotic-treated groups.

Locus coeruleus. The ANOVA revealed a significant effect of treatment on the number of recruited Fos+ neurons in the OXY group (F2,24=6.79, p<0.005) and WD group (F2,26=17.7, p<0.001). The Student-Newman-Keuls post hoc test revealed a significant increase in the number of Fos+ neurons in the OXY+ABX group compared with the OXY group. Example DAB images are shown in FIG. 5B. Example images from the OXY group are repeated from FIGS. 4A-4B for comparisons to the antibiotic-treated groups. A significant difference in the number of Fos+ neurons was found between the SAL, OXY+ABX, WD, and WD+ABX groups.

Periaqueductal gray. The ANOVA revealed a significant effect of treatment on the number of recruited Fos+ neurons in the OXY group (F2,24=32.6, p<0.001) and WD group (F2,26=17.6, p<0.001). The Student-Newman-Keuls post hoc test revealed a significant increase in the number of Fos+ neurons in the OXY+ABX group compared with the OXY group. Example DAB images are shown in FIG. 5B. Example images from the OXY group are repeated from FIGS. 4A-4B for comparisons to the antibiotic-treated groups. The SAL group exhibited a significant decrease in Fos+ neurons compared with all of the other groups. No significant difference in the number of Fos+ neurons was found between the WD and WD+ABX groups.

Lateral habenula medial parvocellular part. The ANOVA revealed no effect of treatment on the number of recruited Fos+ neurons in the OXY group (F2,24=2.71, p<0.87) but a significant effect of treatment in the WD group (F2,26=38.1, p<0.001). The Student-Newman-Keuls post hoc test revealed a significant increase in the number of Fos+ neurons in the WD group compared with the WD+ABX group. Example DAB images are shown in FIG. 6B. Example images from the WD group are repeated from FIGS. 4A-4B for comparison. No difference in the number of Fos+ neurons was found between the SAL, OXY, and OXY+ABX groups (FIGS. 5A-5B). A significant increase in the number of Fos+neurons was observed in the WD and WD+ABX groups compared with the saline group.

Periventricular thalamic nucleus. The ANOVA revealed no effect of treatment on the number of recruited Fos+ neurons in the OXY group (F2,24=1.18, p<0.325) but a significant effect of treatment in the WD group (F2,26=24.1, p<0.001). The Student-Newman-Keuls post hoc test revealed a significant decrease in the number of Fos+ neurons in the SAL group compared with the WD and WD+ABX groups (FIG. 6A). No difference in the number of Fos+ neurons was found between SAL, OXY, and the OXY+ABX groups (FIG. 5A).

Bed nucleus of the stria terminals. The ANOVA revealed no effect of treatment on the number of recruited Fos+ neurons in the OXY group (F2,24=2.24, p<0.129) but a significant effect of treatment in the WD group (F2,26=21.6, p<0.001). The Student-Newman-Keuls post hoc revealed no difference in the number of Fos+ neurons between the SAL, OXY, and OXY+ABX groups (FIG. 5A). The SAL group exhibited a significant decrease in the number of Fos+neurons compared with the WD and WD+ABX groups (FIG. 6A).

Anterior insula. The ANOVA revealed no effect of treatment on the number of recruited Fos+ neurons in the OXY group (F2,24=1.18, p=0.324) and WD group (F2,26=3.87, p=0.034). The Student-Newman-Keuls post hoc test revealed a significant increase in the number of Fos+ neurons in the WD group compared with the SAL group. There was no effect of antibiotics.

### Example 6

### Microbiome Depletion Induced Alterations of Intoxication Network Connectivity

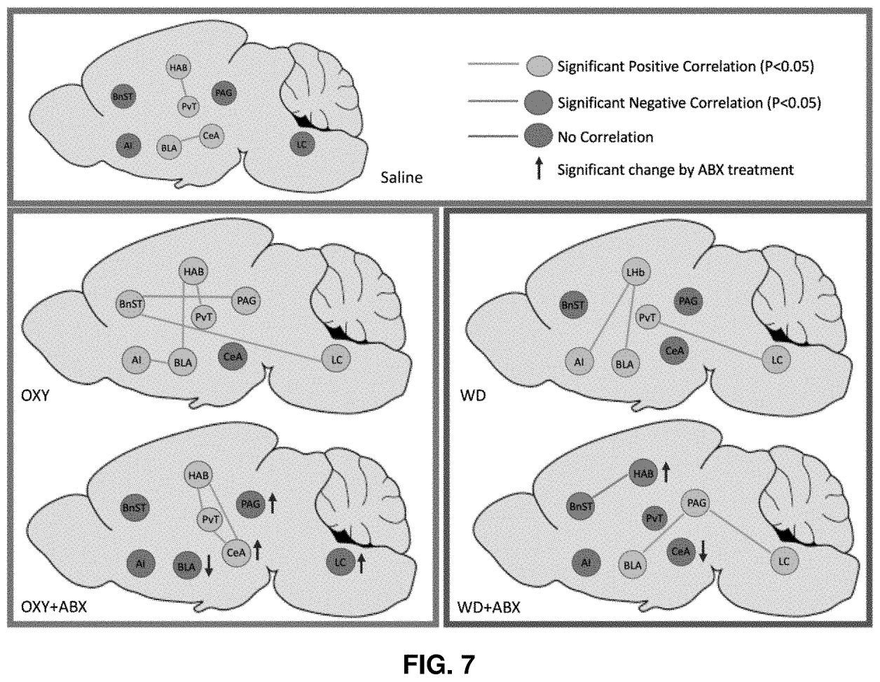

Functional connectivity between brain regions was examined based on significant correlations of Fos+ neurons. Saline control rats that were injected with naloxone exhibited significant positive correlations between the LHb and PVT (R=0.75, p<0.05) and between the BLA and CeA (R=0.78, p<0.05; FIG. 7). During intoxication, oxycodone-dependent rats exhibited an increase in the number of significant positive correlations (five correlations) compared with saline rats (two correlations). In the OXY group, the LHb was positively correlated with the PVT (R=0.78, p<0.05) and BLA (R=0.83, p<0.05). Compared with the SAL group, the OXY group did not exhibit a positive correlation between the BLA and CeA but exhibited a positive correlation between the BLA and AI (R=0.76, p<0.05). The BNST was not correlated with any other region in the SAL group. The BNST was positively correlated with the PAG (R=0.78, p<0.05) and LC (R=0.69, p<0.05) in the OXY group.

In the OXY+ABX group, an overall decrease in positively correlated regions was observed compared with the SAL and OXY groups. Similar to the SAL and OXY groups, the LHb and PVT were positively correlated in the OXY+ABX group (R=0.90, p<0.05). Unlike in the SAL and the OXY groups, the CeA was positively correlated with the LHb (R=0.90, p<0.05), BNST (R=0.76, p<0.05), and PVT (R=0.89, p<0.05) in the OXY+ABX group, which exhibited an overall increase in positive connectivity. The correlation between the BLA and CeA in the SAL group was not present in the OXY+ABX group. Depletion of the microbiome in the OXY+ABX group resulted in a decrease in the number of recruited Fos+ neurons in the BLA (−31%) compared with the matched OXY group. The PAG, CeA, and LC also exhibited 122%, 751%, and 119% increases, respectively, in the number of recruited Fos+ neurons in the OXY+ABX group compared with the OXY group.

### Example 7

### Microbiome Depletion Induced Alterations of Withdrawal Network Connectivity

During withdrawal, the WD group exhibited three positive correlations, contrasting to two positive correlations in the SAL group. In the WD group, the LHb was positively correlated with the BLA (R=0.61, p<0.05) and AI (R=0.62, p<0.05). The PVT was positively correlated with the LC in the WD group (R=0.79, p<0.05). In contrast to the WD group, the WD+ABX group did not exhibit a positive correlation between the LHb and BLA or AI. The WD+ABX group exhibited a negative correlation between the LHb and BNST (R=−0.77, p<0.05). The LHb exhibited a 30% increase in the number of recruited Fos+ neurons, whereas the CeA exhibited a 40% decrease in the number of recruited Fos+neurons in the WD+ABX group compared with the matched WD group. In the WD+ABX group, the PAG was positively correlated with the BLA (R=0.68, p<0.05) and LC (R=0.70, p<0.05), representing a shift in connectivity of the LC from the PVT to the PAG during withdrawal after microbiome depletion in the WD+ABX group.

The statistical table summarized the above examples/studies is provided in Table 4 as follows:

TABLE 4
Statistical Analysis

| FIG. | Data structure | Type of test | Statistical value | p-value |
|---|---|---|---|---|
| FIG. 2A Von Frey Pooled | Two Factors | Two-Way ANOVA | F_{2,44} = 41.0 | <0.001 |
| | (Group and Time | | | |
| | Point) | | | |
| FIG. 2B Withdrawal Score | One factor (treatment) | One-way ANOVA | F_{2,44} = 123.3 | <0.001 |
| Pooled | | Student-Newman-Keuls post hoc | | |
| | | test | | |
| FIG. 2C Von Frey | One factor (treatment) | One-way ANOVA | F_{3,34} = 37.95 | <0.001 |
| Treatment | | Student-Newman-Keuls post hoc | | |
| | | test | | |
| FIG. 2D Withdrawal Score | One factor (treatment) | One-way ANOVA | F_{3,34} = 52.9 | <0.001 |
| | | Student-Newman-Keuls post hoc | | |
| | | test | | |
| FIG. 3A Shannon | One factor (treatment) | One-way ANOVA | F_{2,15} = 2.745 | 0.096 |
| Baseline | | Student-Newman-Keuls post hoc | | |
| | | test | | |
| FIG. 3A Shannon | One factor (treatment) | One-way ANOVA | F_{2,15} = 87.15 | <0.001 |
| Post-Treatment | | Student-Newman-Keuls post hoc | | |
| | | test | | |
| FIG. 3A Chad | One factor (treatment) | One-way ANOVA | F_{2,15} = 1.52 | 0.251 |
| Baseline | One factor (treatment) | Student-Newman-Keuls post hoc | F_{2,15} = 6.873 | <0.008 |
| FIG. 3A Chad | | test | | |
| Post-Treatment | | One-way ANOVA | | |
| | | Student-Newman-Keuls post hoc | | |
| | | test | | |
| Bacteroidetes Post- | One factor (treatment) | One-way ANOVA | F_{2,15} = 8.844 | <0.003 |
| Treatment | | Student-Newman-Keuls post hoc | | |
| | | test | | |
| Firmicutes Post- | One factor (treatment) | One-way ANOVA | F_{2,15} = 27.43 | <0.001 |
| Treatment | | Student-Newman-Keuls post hoc | | |
| | | test | | |
| FIG. 3F Caecum Weight | One factor (treatment) | One-way ANOVA | F_{2,42} = 52.74 | <0.001 |
| FIG. 4A (BLA) | One factor (treatment) | Student-Newman-Keuls post hoc | F_{2,26} = 22.3 | <0.001 |
| | | test | | |
| | | One-way ANOVA | | |
| | | Student-Newman-Keuls post hoc | | |
| | | test | | |
| FIG. 4A (PAG) | One factor (treatment) | One-way ANOVA | F_{2,26} = 16.9 | <0.001 |
| | | Student-Newman-Keuls post hoc | | |
| | | test | | |
| FIG. 4B (LC) | One factor (treatment) | One-way ANOVA | F_{2,26} = 25.2 | <0.001 |
| FIG. 4A (CeA) | One factor (treatment) | Student-Newman-Keuls post hoc | F_{2,26} = 73.97 | <0.001 |
| | | test | | |
| | | One-way ANOVA | | |
| | | Student-Newman-Keuls post hoc | | |
| | | test | | |
| FIG. 4A (PVT) | One factor (treatment) | One-way ANOVA | F_{2,26} = 23 | <0.001 |
| | | Student-Newman-Keuls post hoc | | |
| | | test | | |
| FIG. 4B (Al) | One factor (treatment) | One-way ANOVA | F_{2,26} = 5.92 | <0.001 |
| | | Student-Newman-Keuls post hoc | | |
| | | test | | |
| FIG. 4B (BNST) | One factor (treatment) | One-way ANOVA | F_{2,26} = 55.7 | <0.001 |
| | | Student-Newman-Keuls post hoc | | |
| | | test | | |
| FIG. 4B (LHb) | One factor (treatment) | One-way ANOVA | F_{2,26} = 37.3 | <0.001 |
| | | Student-Newman-Keuls post hoc | | |
| | | test | | |
| FIG. 5A (BLA) | One factor (treatment) | One-way ANOVA | F_{2,24} = 18.8 | <0.001 |
| | | Student-Newman-Keuls post hoc | | |
| | | test | | |
| FIG. 5A (PAG) | One factor (treatment) | One-way ANOVA | F_{2,24} = 32.6 | <0.001 |
| FIG. 5A (LC) | One factor (treatment) | Student-Newman-Keuls post hoc | F_{2,24} = 6.79 | <0.005 |
| | | test | | |
| | | One-way ANOVA | | |
| | | Student-Newman-Keuls post hoc | | |
| | | test | | |
| FIG. 5A (CeA) | One factor (treatment) | One-way ANOVA | F_{2,24} = 5.13 | <0.014 |
| | | Student-Newman-Keuls post hoc | | |
| | | test | | |
| FIG. 5A (PVT) | One factor (treatment) | One-way ANOVA | F_{2,24} = 1.18 | <0.325 |
| FIG. 5A (Al) | One factor (treatment) | Student-Newman-Keuls post hoc | F_{2,24} = 1.18 | <0.324 |
| | | test | | |
| | | One-way ANOVA | | |
| | | Student-Newman-Keuls post hoc | | |
| | | test | | |
| FIG. 5A (BNST) | One factor (treatment) | One-way ANOVA | F_{2,24} = 2.24 | <0.129 |
| | | Student-Newman-Keuls post hoc | | |
| | | test | | |
| FIG. 5A (LHb) | One factor (treatment) | One-way ANOVA | F_{2,24} = 2.71 | <0.87 |
| | | Student-Newman-Keuls post hoc | | |
| | | test | | |
| FIG. 6A (BLA) | One factor (treatment) | One-way ANOVA | F_{2,26} = 2.45 | <0.106 |
| | | Student-Newman-Keuls post hoc | | |
| | | test | | |
| FIG. 6A (PAG) | One factor (treatment) | One-way ANOVA | F_{2,26} = 17.6 | <0.001 |
| | | Student-Newman-Keuls post hoc | | |
| | | test | | |
| FIG. 6A (LC) | One factor (treatment) | One-way ANOVA | F_{2,26} = 17.7 | <0.001 |
| | | Student-Newman-Keuls post hoc | | |
| | | test | | |
| FIG. 6A (CeA) | One factor (treatment) | One-way ANOVA | F_{2,26} = 17.9 | <0.001 |
| FIG. 6A (PVT) | One factor (treatment) | Student-Newman-Keuls post hoc | F_{2,26} = 24.1 | <0.001 |
| | | test | | |
| | | One-way ANOVA | | |
| | | Student-Newman-Keuls post hoc | | |
| | | test | | |
| FIG. 6A (Al) | One factor (treatment) | One-way ANOVA | F_{2,26} = 3.87 | <0.034 |
| | | Student-Newman-Keuls post hoc | | |
| | | test | | |
| FIG. 6A (BNST) | One factor (treatment) | One-way ANOVA | F_{2,26} = 21.6 | <0.001 |
| FIG. 6A (LHb) | One factor (treatment) | Student-Newman-Keuls post hoc | F_{2,26} = 38.1 | <0.001 |
| | | test | | |
| | | One-way ANOVA | | |
| | | Student-Newman-Keuls post hoc | | |
| | | test | | |

### Example 8

### GPR41/43 Co-Expression in Active Ensembles

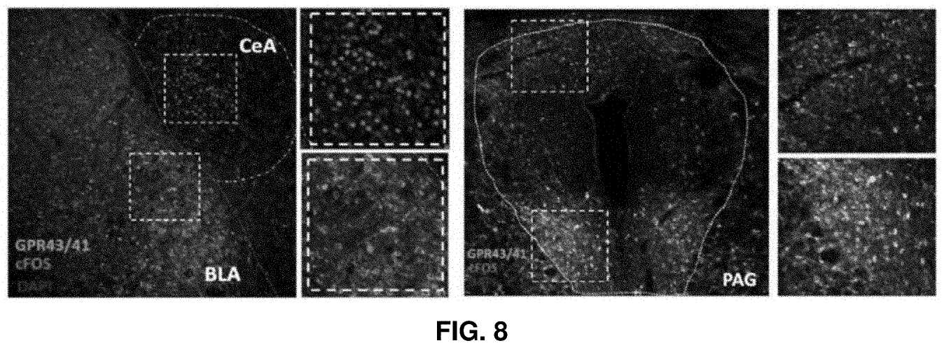

Previous studies have identified the CeA, BLA, and PAG as three structures effected by the depletion of the microbiome. During withdrawal, both the CeA and PAG exhibit an increase in recruited neurons during the intoxication sate in antibiotic treated animals. The BLA exhibits a decrease in recruited neurons in antibiotic treated animals during intoxication. During withdrawal, the CeA exhibits a reduction in recruited ensembles, and there is no change in the PAG or the BLA. Here, GPR41/43 and cFOS were stained during precipitated withdrawal in the same paradigm as the spontaneously withdrawal animals. In the PAG and BLA there is an 89% and 92% overlap respectively between GPR41/43 and cFOS. There is no overlap between GPR41/43 and cFOS in the CeA. While there is no staining in the CeA, it is widely connected to other regions of the extended amygdala, such as the BLA which locally signal during withdrawal (see FIG. 8).

### Example 9

### Depletion of the Microbiome Increases Defensive Irritability-Like Behaviors in the Copenhagen Rat. SCFA Decrease Aggressive Behaviors and Defensive Behaviors During Withdrawal

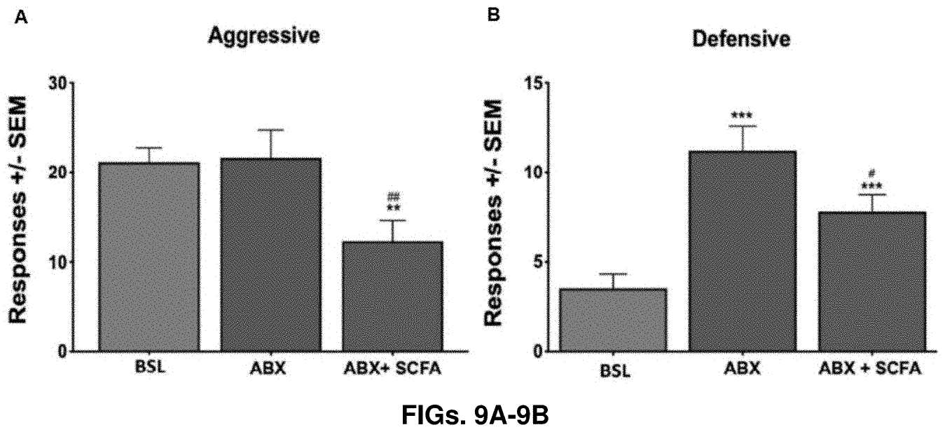

The mixed model RM-ANOVA revealed a significant decrease in aggressive behaviors recorded during the bottle brush irritability test (F2.2, 15.03=8.53, p=0.003) (FIGS. 9A-9B). The Tukey post-hoc test indicated a significant decrease in aggressive behaviors the ABX+SCFA time point relative to the ABX and escalated time point (p's<0.05). The mixed model RM-ANOVA revealed a significant effect of treatment (F1.35, 10.81=26.71, p<0.001). The Tukey post-hoc revealed the defensive behaviors increased following antibiotics treatment (ABX) relative to both the escalated (p<0.001), and ABX+SCFA time point (p<0.05). The ABX+SCFA time point was also increased relative to the escalated time point (p<0.001).

### Example 10

### Depletion of the Microbiome Decreases Pain Threshold During Antibiotic Treatment which is Rescued Upon SCFA Application During Oxycodone Withdrawal in Copenhagen Rats

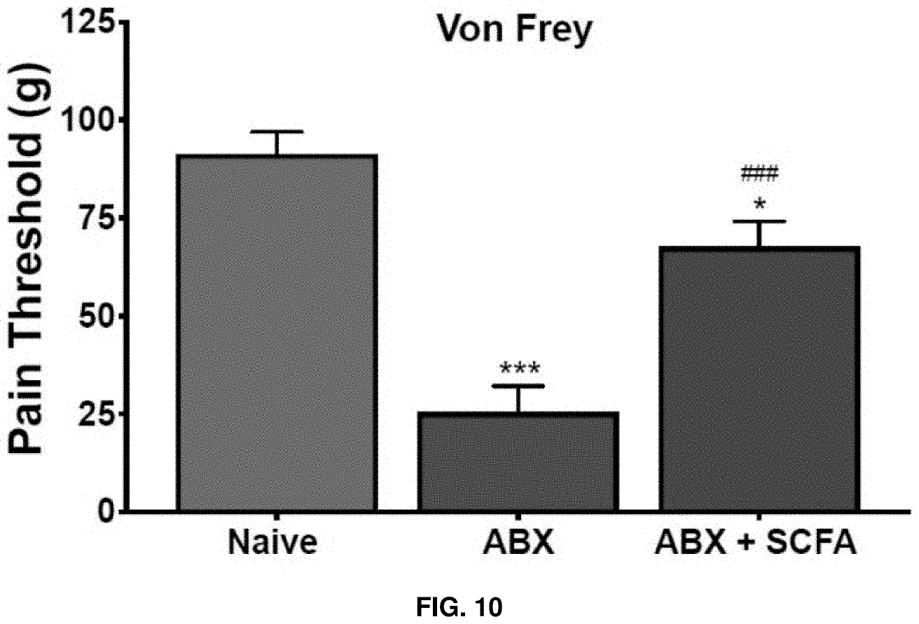

The Brown-Forsythe ANOVA revealed a significant change in von Frey pain threshold after treatment (F2, 15.92=28.05) (p<0.001), see FIG. 10. The Dunnett T3 post-hoc test revealed a decrease at the ABX time point compared to the drug naïve time point (p<0.001), as well as an increase in threshold at the ABX+SCFA time point compared to the ABX time point (p<0.001). The ABX+SCFA time point was also decreased compared to the drug naïve time point (p<0.044).

### Example 11

### Experimental Design

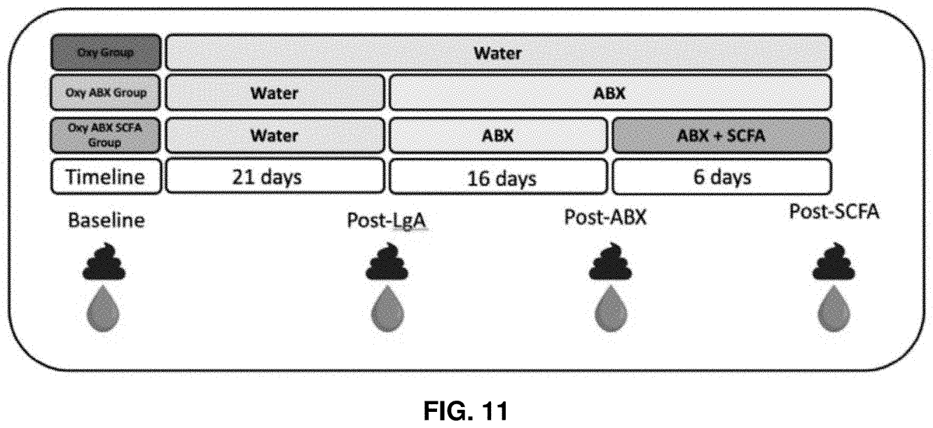

FIG. 11 shows Heterogenous Stock microbiome depletion experimental design. For this experiment, there are four sampling time points for feces and plasma. Baseline sampling is prior to drug and antibiotic exposure to allow for the innate microbiome to be assessed. The Post-LGA time point is following 21 days of oxycodone self-administration. Following the Post-LGA time point, animals were evenly distributed into three groups based on responding—⅓ of the animals received normal drinking water and ⅔ received the antibiotic cocktail. At the third sampling time point, Post-ABX, animals were again split, ½ of the antibiotic treated animals continued to receive the antibiotic cocktail in the drinking water, and ½ were given the antibiotic cocktail+SCFA. Feces and plasma were again harvested following this time point. This separated the animals into three groups, the OXY group, which received normal drinking water the entire experiment, the OXY+ABX group, which received only the antibiotic cocktail following the Post-LGA time point, and the OXY+ABX+SCFA group, which received all three treatments.

### Example 12

### A Subpopulation of Animals Increases Oxycodone Self-Administration Following Antibiotic Depletion of the Microbiome

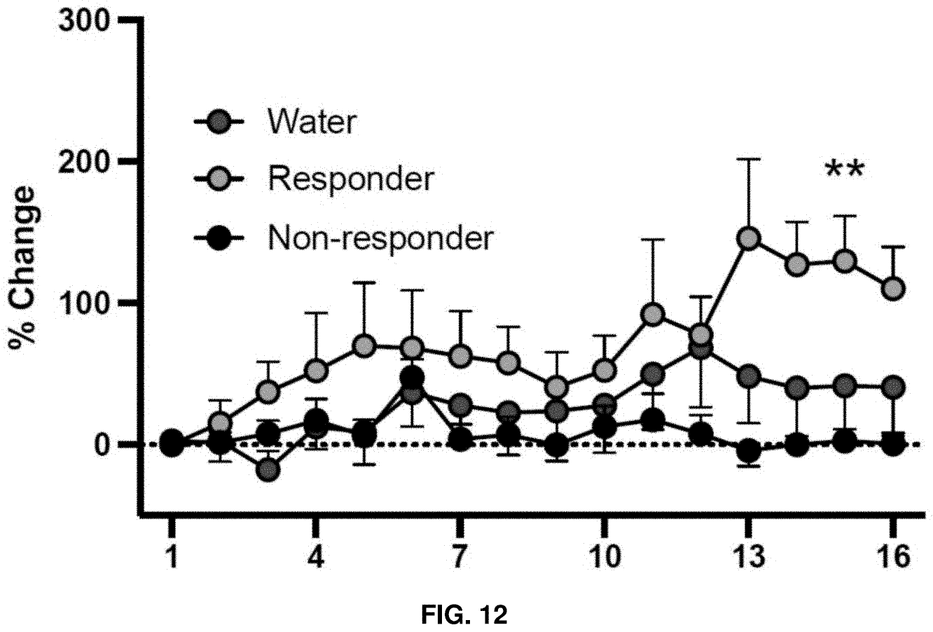

Since there is considerable variation in the HS population of rats, they are plotted as a percent change from an average of the last three days of responding in the long access phase before antibiotics treatment to normalize the magnitude of the observed change (FIG. 12). Animals were considered Responders if there was an increase from the average of the last three days of long access versus the last three days of the antibiotic treatment. A Kolmogorov-Smirnov test revealed that the Responder animals take significantly more oxycodone than the untreated water group (p=0.001, D=0.6875) and Non-Responder groups (p<0.001, D=0.8125).

### Example 13

### Short Chain Fatty Acid (SCFA) Responders

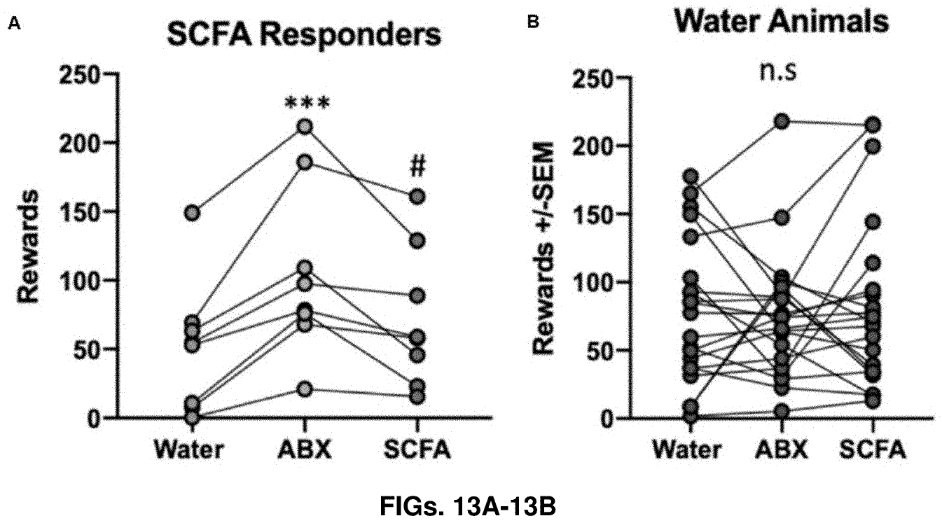

The average of the last three days of long access self-administration before antibiotic treatment (Water), following antibiotic treatment (ABX), and following SCFA treatment (SCFA) was compared. A one-way ANOVA revealed an effect of treatment, (F1.8, 12.71=11.87, p=0.001), individuals increased their intake at the ABX time point compared to the untreated water time point (p=0.003). Individuals also decreased intake following SCFA treatment (p=0.033) The water and Non-Responder animals did not increase their intake at the matched ABX and SCFA time points (p's >0.05), see also FIGS. 13A-13B.

### Example 14

### Untargeted Metabolomics

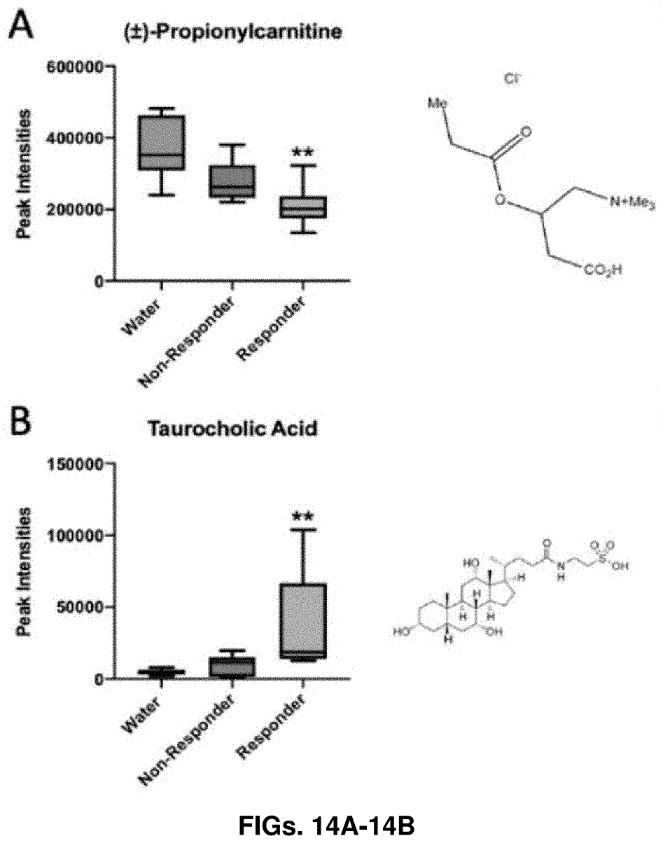

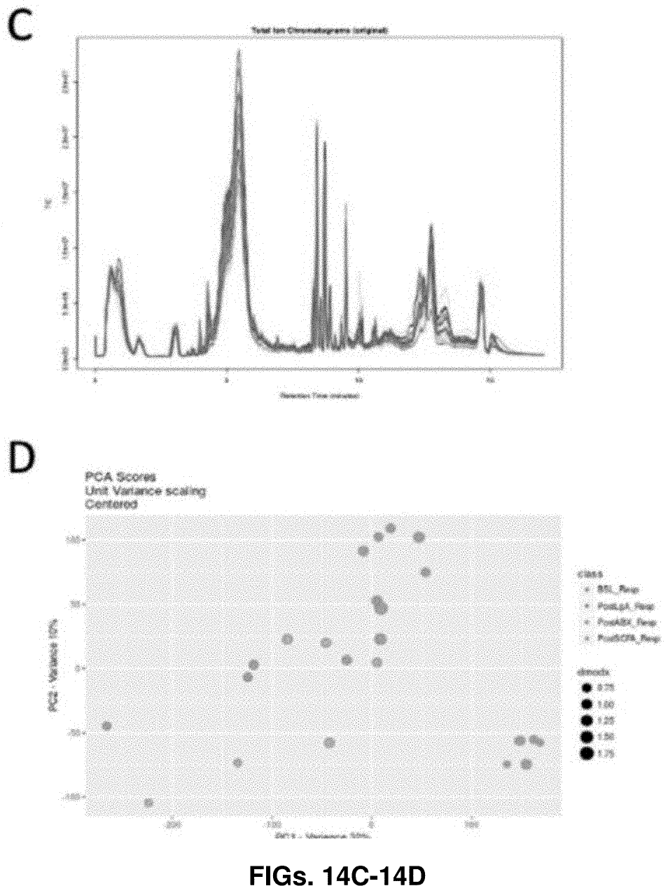

Analysis of circulating plasma using an untargeted metabolomics approach revealed differentially expressed metabolites in Responder and Non-Responder groups. Responders exhibited reduced levels of (±)-propionylcarnitine versus the water group (p=0.005). The Non-Responder group exhibited an intermediate phenotype that did not reach significance compared to the water and the Responder group. The Responder group also exhibited altered levels of Taurocholic acid (p=0.004). According to the post hoc statistical analysis, Responders exhibited an increase in Taurocholic acid compared to the water (p=0.009) but not the Non-Responder groups (p=0.12), see also FIGS. 14A-14D.

### REFERENCES

Aizawa, H. et al (2012). “Molecular characterization of the subnuclei in rat habenula.” Journal of Comparative Neurology 520(18): 4051-4066.
Avery, S. N., et al (2016). “The Human BNST: Functional Role in Anxiety and Addiction.” Neuropsychopharmacology 41(1): 126-141.
Baxley, C., J. et al (2019). “The influence of anxiety sensitivity on opioid use disorder treatment outcomes.” Exp Clin Psychopharmacol 27(1): 64-77.
Belheouane, M., et al (2017). “Improved detection of gene-microbe interactions in the mouse skin microbiota using high-resolution QTL mapping of 16S rRNA transcripts.” Microbiome 5(1): 59.
Brooks, S. P., et al (2003). “Culture-independent phylogenetic analysis of the faecal flora of the rat.” Can J Microbiol 49(10): 589-601.
Bullitt, E. (1990). “Expression of C-fos-like protein as a marker for neuronal activity following noxious stimulation in the rat.” The Journal of Comparative Neurology 296(4): 517-530. CDC/NCHS (2017). National Vital Statistics System.
Chen, J. S., et al (2003). “Short-chain fatty acid inhibitors of histone deacetylases: promising anticancer therapeutics?” Curr Cancer Drug Targets 3(3): 219-236.
Chieng, B. et al (1996). “Local Opioid Withdrawal in Rat Single Periaqueductal Gray Neurons In Vitro.” The Journal of Neuroscience 16(22): 7128-7136.
Chung, L. (2015). “A Brief Introduction to the Transduction of Neural Activity into Fos Signal.” Development & reproduction 19(2): 61-67.
Cicero, T. J., et al (2002). “Gender-linked differences in the expression of physical dependence in the rat.” Pharmacology Biochemistry and Behavior 72(3): 691-697.
Contarino, A. et al (2005). “The corticotropin-releasing factor receptor-1 pathway mediates the negative affective states of opiate withdrawal.” Proc Natl Acad Sci U.S.A. 102(51): 18649-18654.
de Guglielmo, G., et al (2016). “Recruitment of a Neuronal Ensemble in the Central Nucleus of the Amygdala Is Required for Alcohol Dependence.” The Journal of Neuroscience 36(36): 9446-9453.
de Guglielmo, G., et al (2019). “Inactivation of a CRF-dependent amygdalofugal pathway reverses addiction-like behaviors in alcohol-dependent rats.” Nature Communications 10(1): 1238.
den Besten, G., et al (2013). “The role of short-chain fatty acids in the interplay between diet, gut microbiota, and host energy metabolism.” J Lipid Res 54(9): 2325-2340.
Dixon, W. (1965). “The up-and-down method for small samples.” Journal of American Statistical Association (60): 967-978.
Fellows, R., et al (2018). “Microbiota derived short chain fatty acids promote histone crotonylation in the colon through histone deacetylases.” Nat Commun 9(1): 105.
Funk, C. K., et al (2006). “Corticotropin-Releasing Factor within the Central Nucleus of the Amygdala Mediates Enhanced Ethanol Self-Administration in Withdrawn, Ethanol-Dependent Rats.” Journal of Neuroscience 26(44): 11324-11332.
Ge, X., et al (2017). “Antibiotics-induced depletion of mice microbiota induces changes in host serotonin biosynthesis and intestinal motility.” Journal of Translational Medicine 15(1).
Gellert, V. F. et al (1978). “Development and maintenance of morphine tolerance and dependence in the rat by scheduled access to morphine drinking solutions.” Journal of Pharmacology and Experimental Therapeutics 205(3): 536.
George, O., et al (2007). “CRF CRF1 system activation mediates withdrawal-induced increases in nicotine self-administration in nicotine-dependent rats.” Proceedings of the National Academy of Sciences 104(43): 17198-17203.
Giardino, W. J., et al (2018). “Parallel circuits from the bed nuclei of stria terminals to the lateral hypothalamus drive opposing emotional states.” Nature Neuroscience 21(8): 1084-1095.
Gonzalez, A., et al (2018). “Qiita: rapid, web-enabled microbiome meta-analysis.” Nature Methods 15(10): 796-798.
Gracy, K. (2001). “Opiate Withdrawal-Induced Fos Immunoreactivity in the Rat Extended Amygdala Parallels the Development of Conditioned Place Aversion.” Neuropsychopharmacology 24(2): 152-160.
Haight, J. L. et al (2014). “A potential role for the paraventricular nucleus of the thalamus in mediating individual variation in Pavlovian conditioned responses.” 8.
Hikosaka, O., et al (2008). “Habenula: Crossroad between the Basal Ganglia and the Limbic System.” Journal of Neuroscience 28(46): 11825-11829.
Inoue, D., et al (2012). “Short-chain fatty acid receptor GPR41-mediated activation of sympathetic neurons involves synapsin 2b phosphorylation.” 586(10): 1547-1554.
Iredale, P. A., et al (2001). “Role of Corticotropin-Releasing Factor Receptor-1 in Opiate Withdrawal.” Journal of Neurochemistry 74(1): 199-208.
Ivanov A et al (2001). “Local Opiate Withdrawal in Locus Coeruleus Neurons In Vitro” American Physiological Society 85(6): 2388.
Janiri, L. et al. (2009) “Acetyl-L-carnitine in the management of pain during methadone withdrawal syndrome.” Clin Neuropharmacol 32, 35-40.
Kennedy, E. A., et al (2018). “Mouse Microbiota Models: Comparing Germ-Free Mice and Antibiotics Treatment as Tools for Modifying Gut Bacteria.” Frontiers in Physiology 9.
Ketwaroo, G. A., et al (2013). “Opioid-Induced Bowel Dysfunction.” Current Gastroenterology Reports 15(9).
Kimura, I., D. et al (2014). “The SCFA Receptor GPR43 and Energy Metabolism.” Front Endocrinol (Lausanne) 5: 85.
Kimura, I., et al (2011). “Short-chain fatty acids and ketones directly regulate sympathetic nervous system via G protein-coupled receptor 41 (GPR41).” Proceedings of the National Academy of Sciences 108(19): 8030-8035.
Kiraly, D. D., et al (2016). “Alterations of the Host Microbiome Affect Behavioral Responses to Cocaine.” Sci Rep 6: 35455.
Kononoff, J., et al (2018). “Systemic and Intra-Habenular Activation of the Orphan G Protein-Coupled Receptor GPR139 Decreases Compulsive-like Alcohol Drinking and Hyperalgesia in Alcohol-Dependent Rats.” eneuro: ENEURO.0153-0118.
Koob, G. F. (2008). “A Role for Brain Stress Systems in Addiction.” Neuron 59(1): 11-34.
Koob, G. F. (2009). “Brain stress systems in the amygdala and addiction.” Brain Research 1293: 61-75.
Koob, G. F. et al (1997). “Drug Abuse: Hedonic Homeostatic Dysregulation.” Science 278(5335): 52.
Koya, E., et al (2012). “Silent synapses in selectively activated nucleus accumbens neurons following cocaine sensitization.” Nature Neuroscience 15(11): 1556-1562.
Kreibich, A., et al (2008). “Presynaptic Inhibition of Diverse Afferents to the Locus Ceruleus by -Opiate Receptors: A Novel Mechanism for Regulating the Central Norepinephrine System.” Journal of Neuroscience 28(25): 6516-6525.
Le Bastard, Q., et al (2018). “Systematic review: human gut dysbiosis induced by non-antibiotic prescription medications.” Alimentary Pharmacology & Therapeutics 47(3): 332-345.
Lee, K., et al (2018). “The gut microbiota mediates reward and sensory responses associated with regimen-selective morphine dependence.” Neuropsychopharmacology 43(13): 2606-2614.
Leppert, W. (2012). “The impact of opioid analgesics on the gastrointestinal tract function and the current management possibilities” Wspótczesna Onkologia 2: 125-139.
Louis, P. et al (2017). “Formation of propionate and butyrate by the human colonic microbiota.” Environ Microbiol 19(1): 29-41.
Martinotti, G. et al. (2010) “Acetyl-L-carnitine for alcohol craving and relapse prevention in anhedonic alcoholics: a randomized, double-blind, placebo-controlled pilot trial.” Alcohol 45, 449-455.
Meng, J., et al (2015). “Opioid Exacerbation of Gram-positive sepsis, induced by Gut Microbial Modulation, is Rescued by IL-17A Neutralization.” Scientific Reports 5: 10918.
Morrison D, (2016). “Formation of short chain fatty acids by the gut microbiota and their impact on human metabolism.” Gut Microbes 7(3): 11.
Paxinos, G., et al. (2007). The Rat Brain in Stereotaxic Coordinates Amsterdam Elsevier.
Proulx, C. D., et al (2014). “Reward processing by the lateral habenula in normal and depressive behaviors.” Nature Neuroscience 17(9): 1146-1152.
Reikvam, D. H., et al (2011). “Depletion of Murine Intestinal Microbiota: Effects on Gut Mucosa and Epithelial Gene Expression.” PLOS ONE 6(3): e17996.
Sartorius, A. et al (2007). “Deep brain stimulation of the lateral habenula in treatment resistant major depression.” Medical Hypotheses 69(6): 1305-1308.
Schindelin, J., et al (2012). “Fiji: an open-source platform for biological-image analysis.” Nature Methods 9(7): 676-682.
Shabel, S. J., et al (2014). “GABA/glutamate co-release controls habenula output and is modified by antidepressant treatment.” Science 345(6203): 1494-1498.
Tye, K. M., et al (2011). “Amygdala circuitry mediating reversible and bidirectional control of anxiety.” Nature 471(7338): 358-362.
Van Bockstaele, E. J., et al (2006). “Amygdaloid Corticotropin-Releasing Factor Targets Locus Coeruleus Dendrites: Substrate for the Co-ordination of Emotional and Cognitive Limbs of the Stress Response.” Journal of Neuroendocrinology 10(10): 743-758.
Van Bockstaele, E. J., et al (2010). “The locus coeruleus: A key nucleus where stress and opioids intersect to mediate vulnerability to opiate abuse.” Brain Research 1314: 162-174.
Van De Wouw, M., et al (2018). “Short-chain fatty acids: microbial metabolites that alleviate stress-induced brain-gut axis alterations.” The Journal of Physiology 596(20): 4923-4944.
van de Wouw, M., et al (2018). “Short-chain fatty acids: microbial metabolites that alleviate stress-induced brain-gut axis alterations.” J Physiol 596(20): 4923-4944.
Vinolo, M. A., et al (2011). “Regulation of inflammation by short chain fatty acids.” Nutrients 3(10): 858-876.
Waldecker, M., et al (2008). “Inhibition of histone-deacetylase activity by short-chain fatty acids and some polyphenol metabolites formed in the colon.” J Nutr Biochem 19(9): 587-593.
Wang, F., et al (2018). “Morphine induces changes in the gut microbiome and metabolome in a morphine dependence model.” Scientific Reports 8(1).
Wassum, K. M. et al (2015). “The basolateral amygdala in reward learning and addiction.” Neuroscience Biobehavioral Research 57: 271-283.
Wiebelhaus, J. M., et al (2015). “Effects of Acute and Repeated Administration of Oxycodone and Naloxone-Precipitated Withdrawal on Intracranial Self-Stimulation in Rats.” Journal of Pharmacology and Experimental Therapeutics 356(1): 43-52.

The preceding disclosure and/or examples are offered for illustrative purposes only and are not intended to limit the scope of the invention in any way. Various modifications of the invention in addition to those shown and described herein will become apparent to those skilled in the art from the foregoing description and fall within the scope of the appended claims.

1. A composition comprising a therapeutically effective amount of a carnitine derivative and a therapeutically effective amount of a short chain fatty acid.

2. The composition of claim 1, wherein the carnitine derivative comprises L-carnitine, L-carnitine-L-tartrate, 2-methylbutyrylcarnitine, acetylcarnitine, butyrylcarnitine, carnitine, decanoylcarnitine, hexanoylcarnitine, isobutyrylcarnitine, isovalerylcarnitine, lauroylcarnitine, myristoylcarnitine, octanoylcarnitine, palmitoylcarnitine, propionylcarnitine, stearoylcarnitine, valerylcarnitine, or any combination thereof.

3. The composition of claim 2, wherein the therapeutically effective amount of the carnitine derivative comprises from about 400 mg/day to about 4000 mg/day.

4.-7. (canceled)

8. The composition of claim 1, wherein the short chain fatty acid comprises acetic acid, propionic acid, butyric acid, isobutyric acid, valeric acid, isovaleric acid, or any combination thereof.

9. The composition of claim 8, wherein the therapeutically effective amount of the short chain fatty acid is from about 500 mg/day to about 4000 mg/day.

10. The composition of claim 1, wherein the short chain fatty acid comprises butyric acid or propionic acid and the carnitine derivative comprises propionylcarnitine.

11. The composition of claim 1, wherein the composition is non-addictive

12. An oral dosage form comprising the composition of claim 1 and a pharmaceutically acceptable excipient.

13.-14. (canceled)

15. A method for treating or preventing a disease or disorder in a subject, the method comprising administering the composition of claim 1 to the subject.

16. The method of claim 15, wherein the disease or disorder comprises addiction to an opioid, cocaine addiction, alcohol addiction, nicotine addiction, depression, anxiety, pain, a metabolic disorder, or any combination thereof, and wherein the disease or disorder relates to restore a population of at least one microbial species in the subject's gastrointestinal tract after the subject has taken antibiotics.

17.-29. (canceled)

30. The method of claim 15, wherein the method increases a population of at least one microbial species in the subject's gastrointestinal tract.

31. The method of claim 30, wherein the at least one microbial species comprises a Bacterioides species, a Firmicutes species, a Clostridium species, a Eubacterium species, a Ruminococcus species, a Fusobacterium species, a Burkholderia species, a Butyrivibrio species, a Prevotella species, an Agrobacterium species, a Desulfovibrio species, a Gammaproteobacteria species, an Enterobacteriaceae species, a Eubacteriaceae species, an Anaerofustis species, a Turicibacter species, an Elusimicrobiaceae species, a Bifidobacterium species, a Lactobacillus species, Akkermansia mucinophila, Megasphaera elsdenii, Mitsuokella multiacida, Roseburia intestinalis, Faecalibacterium prausnitzii, Veillonella parvula, Acinetobacter calcoaceticus, Pseudomonas aeruginosa, Biophila wadsworthia, or any combination thereof.

32.-36. (canceled)

37. The method of claim 15, wherein the method reduces Fos expression in at least one region of the brain comprising the basolateral amygdala, central amygdala, periaqueductal gray matter, locus coeruleus, lateral habenula, paraventricular thalamic nucleus, anterior insula, bed nucleus of the stria terminalus, or any combination thereof.

38.-46. (canceled)

47. A method for diagnosing addiction to at least one substance in a subject, the method comprising:
a. obtaining a biological sample from the subject; and
b. measuring a level of at least one metabolite in the biological sample.

48. The method of claim 47, wherein the at least one substance comprises an opioid selected from the group consisting of oxycodone, heroin, fentanyl, or any combination thereof, cocaine, alcohol, nicotine, or any combination thereof, and wherein the biological sample comprises whole blood, plasma, serum, bile, urine, feces, or any combination thereof.

49.-50. (canceled)

51. The method of claim 47, wherein the at least one metabolite comprises (±)-propionylcarnitine, taurocholic acid, or any combination thereof, and wherein a subject having addiction has a lower level of the at least one metabolite compared to a non-addicted subject.

52.-53. (canceled)

54. A method of monitoring the progress of an addiction treatment in a subject diagnosed with an addiction, the method comprising:
a. obtaining a first biological sample from the subject prior to initiating the addiction treatment;
b. measuring a level of at least one metabolite in the first biological sample;
c. initiating the addiction treatment;
d. obtaining a second biological sample from the subject;
e. measuring a level of the at least one metabolite in the second biological sample; and
f. comparing the level of the at least one metabolite in the first biological sample to the level of the at least one metabolite in the second biological sample;
wherein an increased level of the at least one metabolite in the second biological sample compared to the level of the at least one metabolite in the first biological sample indicates the addiction treatment is succeeding.

55. The method of claim 54, wherein the first biological sample and the second biological sample comprise whole blood, plasma, serum, bile, urine, feces, or any combination thereof.

56. The method of claim 54, wherein the at least one metabolite comprises (±)-propionylcarnitine, taurocholic acid, or any combination thereof.

57. (canceled)

58. The method of claim 54, wherein the addiction treatment comprises administering the composition of claim 1.
